(19) United States
(12) Patent Application Publication
(10) Pub. No.: US 2006/0194815 A1
(43) Pub. Date: Aug. 31, 2006

(54) Methods and compositions for modulating serum cortisol levels

(76) Inventors: Laurent Lecanu, McLean, VA (US); Janet Greeson, Las Vegas, NV (US); Vassilios Papadopoulos, North Potomac, MD (US); Jing Xu, Rockville, MD (US)

Correspondence Address: Schwegman, Lundberg, Woessner & Kluth, P.A., P.O. Box 2938, Minneapolis, MN, 55402, US

(21) Appl. No.: 11/293,866

(22) Filed: Dec. 2, 2005

Related U.S. Application Data
(63) Continuation of application No. PCT/US04/16126, filed on May 20, 2004.
(60) Provisional application No. 60/474,964, filed on Jun. 2, 2003.
(60) Provisional application No. 60/478,648, filed on Aug. 1, 2003.
(60) Provisional application No. 60/564,636, filed on Apr. 22, 2004.
(60) Provisional application No. 60/475,642, filed on Jun. 4, 2003.
(60) Provisional application No. 60/566,869, filed on Apr. 30, 2004.

(51) Int. Cl. A61K 31/496

(52) U.S. Cl. 514/254.09

(57) ABSTRACT

The present invention relates to cortisol-modulating compounds, including but not limited to benzamide and benzoic acid derivatives such as procaine and procaine derivatives, utilized in compositions and methods for treating cortisol-mediated disorders, including but not limited to age-related depression, hypertension, Alzheimer's disease, and acquired immunodeficiency syndrome.

### FIELD OF THE INVENTION

The present invention is related to a pharmaceutical composition containing a cortisol-modulating agent; to the use of such a composition in treating disease; to combinations with such a composition with other therapeutic agents; and to kits containing such a composition.

### BACKGROUND OF THE INVENTION

Cortisol, a glucocorticoid hormone secreted by the adrenal cortex, is responsible for the regulation of fat, carbohydrate, and protein metabolism. In humans, cortisol is the main circulating glucocorticoid and it is involved in different physiological functions such as sleep cycle regulation, metabolism, immunity, mood normalization, memorization and leaning. Variations in plasma cortisol concentration are also associated with diseases of the musculoskeletal gastrointestinal, cardiovascular, endocrine and central nervous systems. Excessive cortisol synthesis leads to changes in metabolism, cognitive impairment (McEwen, 1994) and immunosuppression (Chrousos and Gold, 1992). Abnormalities at different levels of the hypothalamic-pituitary-adrenal (HPA) axis have been reported in several diseases, such as psychiatric disorders, including depression and mood alteration (Kiraly et al., 1997; Tafet et al., 2001), acquired immunodeficiency syndrome (AIDS) (Corley, 1996; Bhansali et al., 2000; Christeff et al., 2000), multiple sclerosis (Erkut et al., 2002), dementia (Maeda et al., 1991; Polleri et al., 2002), Alzheimer's disease (Swaab et al., 1994; O'Brien et al., 1996; Weiner et al., 1997; Giubilei et al., 2001; Rasmuson et al., 2002), and breast cancer outcome (Luecken et al., 2002). Disruption of hormonal balance in these diseases leads to increased cortisol production resulting in elevated concentrations of cortisol in cerebrospinal fluid (Swaab et al., 1994; Erkut et al., 2002), blood (Weiner et al., 1997; Bhansali et al., 2000; Rasmuson et al., 2002), urine (Maeda et al., 1991) and saliva (Giubilei et al., 2001).

At the cellular level, glucocorticoids such as cortisol have been shown to decrease cytochrome c oxidase activity (Simon et al., 1998) and to induce apoptosis in various cell lines (Montague and Cidlowski, 1995). Cortisol is derived from cholesterol. Steroidogenesis begins with the mobilization of free cholesterol and transport from intracellular stores into mitochondria where cholesterol will be metabolized into pregnenolone by the first enzyme of the pathway, the cytochrome P-450 side-chain cleavage enzyme complex (P-450_{SCC}). Hormones, such as corticotrophin (ACTH) and its second messenger cAMP (adenosine 3′,5′-cyclic phosphate), acting through the cAMP-dependent protein kinase (PKA), accelerate this process. Although cholesterol transport into mitochondria is the rate-determining step in steroid biosynthesis, steroid formation is also limited by the amount of the substrate cholesterol available. Cholesterol availability depends on the rate of its synthesis and thus, the activity of the rate-limiting enzyme 3-hydroxy-3-methylglutaryl-coenzyme A (HMG-CoA) reductase located in the cytoplasm, responsible for the conversion of HMG-CoA to mevalonate.

Hypercholesterolemia is one of the main reasons for various pathologies of the cardiovascular system. At present, statins are used as the major therapeutic means for hypercholesterolemia because they occupy a portion of the binding site of HMG-CoA, thus blocking access of this substrate to the active site of HMG-CoA reductase (Istvan and Deisenhofer, 2001). In addition, statins are in clinical trials for their use to slow Alzheimer's disease progression, a disease where hypercholesterolemia seems to play a critical role (Waldman and Kitharides, 2003). However, it has been recently reported that statins have numerous serious side effects. Other proposed therapies focus on blocking the activity of enzymes involved in the steroidogenic pathway. However, such methods and compositions result in the inhibition of physiological basal steroid synthesis and may cause detrimental effects to the health of the subject.

HIV-associated dementia (also known as HIV-Associated Dementia Complex, HIV-associated cognitive/motor complex, and AIDS Dementia Complex) is a progressive neurological disorder that affects approximately 58,000 individuals infected with the Human Immunodeficiency Virus (HIV) in the United States. HIV-associated dementia is thought to be a subcortical dementia characterized by cognitive, motor and behavioral impairments severe enough to interfere with an individual's ability to function occupationally or socially. Early manifestations of HIV-associated dementia may be characterized by cognitive impairment, loss of motor skills, and/or behavioral challenges:

Cognitive Impairment: Memory loss, impaired concentration, and mental slowing characterized by such actions as slow response are common attributes associated with cognitive impairment.

Loss of Motor Skills: Individuals experiencing difficulty with their balance, lack of coordination, leg weakness, clumsiness, poor gait, and/or deteriorating handwriting may be showing signs of deteriorating motor skills.

Behavioral Challenges: Uncharacteristic behavior, poor decision-making, personality and mood changes, and possibly psychotic behavior characterize the behavioral challenges experienced by some individuals.

Individuals suffering from HIV-associated dementia may develop these characteristics at various times and rates during the progression of the disease. HIV-associated dementia patients typically experience a high incidence of premature mortality due to or associated with their dementia. Dementia is a debilitating disease that literally steals the livelihood of its victims. Memory loss, depression, agitation, anxiety, and other adverse behaviors are caused by its apparently irreversible and destructive effects on the central nervous system. These debilitating effects further reduce the life expectancy of HIV infected individuals. Working in concert, and without effective treatment, the virus and the dementia condition, destroy individual's immune systems, self-confidence, motor skills, and family relations. As a result, individuals with HIV-associated dementia experience premature mortality.

In the absence of dementia, treatments for HIV affected individuals are given an opportunity to be more effective and possibly prolong the life of the individual. Further, in the absence of this condition the treatment may prove effective in retarding the replication of HIV and retarding its adverse effects.

Increased intracellular calcium concentration in neurons and/or increased cortisol production by the adrenal, induced due to changes in hypothalamus-pituitary-adrenal (HPA) axis and probably glycoprotein 120 (gp 120) may trigger events that lead to HIV-associated dementia. (Corley P A. 1995; Corley P A. 1996; Simpson, David M; Brew, Bruce James 1999). HIV, the virus whose progression leads to Acquired Immune Deficiency Syndrome (AIDS), is a retrovirus housed in a viral particle protected by various coat proteins, the most significant of which is glycoprotein 120 (gp120). The gp120 envelope facilitates infection of a host cell by binding to receptors on the surface of many immune cells such as T-cells as well as chemokine co receptors. After fusion of the viral particle with the host cell, replication of the viral particles is initiated and subsequent infection of other cells occurs.

In addition to facilitating the introduction of HIV into host cells, research demonstrates that gp120 is either directly or indirectly responsible for initiating HIV dementia. The direct hypothesis suggests that the gp120 protein, which is often shed from the HIV virus after fusion occurs, interacts directly with chemokine receptors on the surface of neurons; thereby facilitating apoptosis and neuronal cell death. (Brew, Bruce James 1999). The indirect hypothesis suggests that apoptosis is caused by interaction of the HIV virus with non-neuronal cells of the central nervous system (CNS), specifically macrophages, microglia, and astrocytes. In this case, gp120 facilitates the transport of HIV infected macrophages and microglia across the blood brain barrier (BBB), a selectively permeable membrane that prevents entry of foreign material. (Kaul, Marcus, et al. 2001). Once infected cells are in the brain, they release neurotoxins and promote a massive influx of calcium ion (Ca²⁺) into the neuron thus initiating apoptosis. (Smits, H. A. et al. 2000). HIV Infected macrophages, monocytes and microglia all release gp120. An abundance of gp120 in the CNS disrupts the calcium homeostasis (Lipton S A, 1994) partly by reverting the glutamate uptake systems and by directly activating the NMDA subtype calcium channel-associated glutamatergic receptor and the calcium voltage-operated channels (Lipton S A, 1991). The induced massive calcium inward current leads to an impairment of the memory and learning processes and triggers the excitotoxicity cascade which leads to a neuronal death (Choi D W, 1992). Calcium ions facilitate intercellular communication through electrical polarization and depolarization and therefore opening a Ca²⁺ channel for too long is fatal to a neuron. (Epstein L and Gendelman H. May 1993).

A combination of both the direct and indirect interference of gp120 with the calcium homeostasis may cause mitochondrial function impairment leading to critical cell death. (Simpson, David M.). At the same time, gp120 indirectly induces an increase in blood and CSF cortisol concentrations leading to neurotoxicity and HIV-associated dementia. (Corley P A. 1995; Corley P A. 1996).

Chemokine receptors are also bound by the gp120 envelope as co receptors with CD4 to permit entry into host cells. (Miller, Richard J. and Meucci, Olimpia 1999). This binding on cells of the CNS acts to stimulate and agonize the cells in an uncontrolled manner. Over stimulation subsequently acts to release glutamate and other neurotoxins and inflammatory cytokines resulting in neuronal death due to apoptosis. (Miller, Richard J. and Meucci, Olimpia 1999).

Astrocytosis, proliferation of astrocytes, observed in patients with HIV, occurs when the virus retards the effectiveness of astrocytes to scavenge excess glutamate produced by infected macrophages and microglia. (Kaul, Marcus et al. 2001). Additional astrocytes are produced to compensate for the ineffectiveness of the cells. As a result of astrocytosis, more infected macrophages and microglia cross the BBB inducing massive neuronal death which leads to HIV-associated dementia.

It is clear that the cause of HIV-associated dementia revolves around the activation of macrophages, microglia, chemokine receptors, and astrocytes within the CNS and subsequent apoptosis leading to dementia. It is equally apparent that the process is made possible because the gp120 envelope facilitates transfer of the HIV virus across the BBB and because cleaved gp120 protein is able to interact with chemokine receptors on the surface of neurons. Prevalent theory also posits that HIV-associated elevation of cortisol levels is directly responsible for induction of HIV-associated dementia.

The progression of HIV infection is accompanied by complex alterations in the production of adrenal steroids. HIV infection is associated with activation of the hypothalamic-pituitary-adrenal (HPA) axis function, leading to increased plasma and urinary cortisol levels. (Kumar M, et al. 2001; Kumar, M, et al. 2000). Increased cortisol levels have been documented in both HIV-infected individuals and patients with AIDS. The increases in cortisol levels range from 20% to 50% above normal; the highest levels are found in patients with advanced disease. HIV-associated elevation of cortisol levels is hypothesized to have a major function in the pathophysiology of AIDS, including suppression of cell-mediated immunity and AIDS-associated dementia (Corley P A. 1995). In addition, there is experimental evidence suggesting that cortisol and its receptors are involved in the regulation of immune function in HIV infection. (Norbiato G, et al. 1997).

Refaeli and colleagues have found a different line of evidence linking cortisol to AIDS. These investigators have shown that an HIV protein, the product of the vpr gene, mimics the actions of the glucocorticoids, including cortisol. Previously, it had been shown that the vpr protein pierces the membranes of macrophages, the white cells that are among the first immune cells to host HIV infection. Refaeli's group has provided evidence suggesting that this HIV protein dupes the body into suppressing its own immune system. The vpr protein blocks the production of the Type 1 cytokines. In addition, the vpr protein was shown to induce healthy, uninfected T-lymphocytes in initiating programmed cell death. When these investigators added the steroidogenesis inhibitor, RU-486, to tissue culture cells, they found that it reversed the vpr protein's destructive effects on immunity. Furthermore, T-lymphocytes treated with the vpr protein and RU-486 continued to synthesize and secrete immune-boosting cytokines and did not succumb to programmed cell death.

Thus, HIV-associated elevation of cortisol levels may be implicated in the pathophysiology of AIDS, including suppression of cell-mediated immunity and HIV-associated dementia as suggested by Corley. On the basis of this conclusion, Clerici and associates have postulated that preventing or reversing the cortisol: DHEA ratio and the induction of Type 1 cytokine production in patients with AIDS may serve to reduce programmed cell death and interfere with viral replication in HIV-infected cells.

In contrast to the detrimental effects of high levels of cortisol in the pathologies described above, maintenance of the basal cortisol levels is necessary for the maintenance of basic biological functions. Glucocorticoids regulate the metabolism of proteins, carbohydrates and lipids, and are essential to the adaptation to acute physical stressors (Munck et al, 1994). Development of compounds which block the excessive glucocorticoid synthesis without affecting the basal steroid formation has proven to be a difficult task because it requires the identification of a modulator of an activity rather than an inhibitor. Therefore, there is a need for additional treatments of a cortisol-mediated disease or disorder, including compositions for administration to a subject suffering from, or at risk of developing, a cortisol-mediated disease or disorder. A faster onset of action, improved side-effect profile, reduced dosing amount and frequency, improved patient compliance, improved bioavailability and safety, and/or improved pharmacokinetic, pharmacodynamic, chemical and/or physical properties are also desired for the treatment of such conditions. Additionally, there is a need for methods and compositions that modulate levels of cortisol while maintaining basal cortisol levels. The discussion that follows discloses methods, kits, combinations, and compositions that help to fulfill these needs.

### SUMMARY OF THE INVENTION

The effective treatment of a cortisol-mediated condition, disease, or disorder in a subject has been complicated by the lack of treatment options available, and the lack of efficacy of these currently available options. However, cortisol-modulating agents, for example, benzoic acid derivatives such as procaine and procaine derivatives, have been discovered to down-regulate hormone-induced glucocorticoid formation without affecting basal corticosteroid formation. Pharmaceutical compositions comprising a benzoic acid derivative can effectively deliver to a subject a therapeutically-effective amount of a pharmaceutical agent to treat and/or prevent such cortisol-mediated conditions, diseases, or disorders, including, for example, age-related depression, mood alteration, multiple sclerosis, Cushing's syndrome, hypertension, dementia and Alzheimer's disease, and acquired immunodeficiency syndrome. These compositions provide enhanced treatment options and possess improved efficacy, bioavailability, safety, and/or improved pharmacokinetic, pharmacodynamic, chemical and/or physical properties. The present invention comprises these pharmaceutical compositions, dosage forms and kits based thereon, and methods for the preparation and use thereof.

Further, benzoic acid derivative compounds may be utilized in methods and compositions for modulating cortisol levels in combination with other therapeutic agents.

### BRIEF DESCRIPTION OF THE DRAWINGS

FIG. 1 depicts the chemical formula of several benzoic acid derivatives including procaine and several procaine derivatives of the present invention.

FIG. 2A is a bar graph depicting the effect of procaine and SP-10 on the dbc-AMP-induced 20á-hydroxyprogesterone synthesis in pg/well compared to control in Y1 mouse adrenal tumor cells.

FIG. 2B is a bar graph depicting the effect of procaine and SP-10 on cell viability compared to control in dbc-AMP induced Y1 mouse adrenal tumor cells.

FIG. 2C is a bar graph depicting the effect of SP014, SP016, and SP017 on the dbc-AMP-induced 20á-hydroxyprogesterone synthesis in inhibition percentage in Y1 mouse adrenal tumor cells.

FIG. 2D is a bar graph depicting the effect of SP014, SP016, and SP-17 on cell viability compared to control in dbc-AMP-induced Y1 mouse adrenal tumor cells.

FIG. 3A is a bar graph depicting the effect of procaine on the dbc-AMP-induced cortisol synthesis in H295R human adrenal tumor cells.

FIG. 3B is a bar graph depicting the effect of procaine on cell viability in dbc-AMP-induced H295R human adrenal tumor cells.

FIG. 4A is a bar graph depicting the effect of procaine on the dbc-AMP-induced progesterone synthesis in MA-10 mouse Leydig tumor cells.

FIG. 4B is a bar graph depicting the effect of procaine on cell viability in dbc-AMP-induced MA-10 mouse Leydig tumor cells.

FIG. 5 is graph depicting the effect of a procaine-based formulation on serum corticosterone levels in male Sprague-Dawley rats.

FIG. 6 is a graph depicting the effect of procaine on the dbc-AMP-induced increase of the PKA activity.

FIG. 7A is a bar graph depicting the effect of procaine on hydroxycholesterol induced 20á-hydroxyprogesterone synthesis.

FIG. 7B is an immunoblot depicting the effect of procaine on the dbc-AMP-induced expression of the P450_{SCC} enzyme.

FIG. 7C is an immunoblot depicting the effect of procaine on the dbc-AMP-induced StAR expression.

FIG. 8A is a bar graph depicting the effect of procaine on dbcAMP and mevalonate supported 20á-hydroxyprogesterone formation in Y1 cells.

FIG. 8B is a bar graph depicting the effect of procaine on HMG-CoA reductase activity in Y1 cells treated with dbcAMP (** p<0.01 *** p<0.001, mean±SD). 100% activity corresponds to 163±16 pmol/min/mg protein.

FIG. 9A is bar graph depicting the effect of procaine on HMG-CoA reductase mRNA expression levels by Q-PCR in dbcAMP induced versus control Y1 cells.

FIG. 9B is bar graph depicting the effect of procaine on HMG-CoA reductase mRNA expression levels by Q-PCR in dbcAMP induced versus control UT-1 cells.

FIG. 9C is bar graph depicting the effect of procaine on HMG-CoA reductase mRNA expression levels by Q-PCR in dbcAMP induced versus control Hepa1-6 mouse liver hepatoma cells.

### DETAILED DESCRIPTION

The present invention is directed to methods, kits, combinations, and compositions for treating a disease, condition or disorder where treatment with a cortisol-modulating agent is indicated. It has been discovered that pharmaceutical compositions comprising benzoic acid derivatives, such as procaine or a procaine derivative for example, exhibit superior performance as cortisol-modulating agent containing pharmaceutical compositions. In therapy of a disease, condition or disorder, it is important to provide a dosage form that delivers the required therapeutic amount of the drug in vivo, and renders the drug bioavailable in a rapid manner. The formulations of the present invention satisfy these needs.

While the present invention may be embodied in many different forms, several specific embodiments are discussed herein with the understanding that the present disclosure is to be considered only as an exemplification of the principles of the invention, and it is not intended to limit the invention to the embodiments illustrated.

In one aspect of the present invention, the compositions, methods, kits, combinations are useful for the effective treatment or prevention of a cortisol-mediated disease, condition, or disorder in a subject. In one embodiment, the compositions, methods, kits, combinations are useful for treating or preventing a disease, condition, or disorder associated with, or related to, excessive cortisol syntheses in a subject. Illustratively, the present invention is useful for the treatment or prevention of a cortical-mediated disease including a musculoskeletal, gastrointestinal, cardiovascular, endocrine and/or central nervous system (including, for example, stroke, brain, retina and/or spinal cord injuries, ischemia and reperfusion, and other brain or retinal disorders, and trauma associated with neurosurgical procedures) disease or disorder; a change in metabolism; cognitive impairment; immunosuppression; a psychiatric disorder, including depression and mood alteration; acquired immunodeficiency syndrome; multiple sclerosis; Alzheimer's disease; Huntington's disease; epilepsy; lathyrism; amyotrophic lateral sclerosis; Parkinson's disease; and cancer, including, for example breast cancer.

Definitions

Unless otherwise stated, the following terms used in the specification and claims are defined for the purposes of this application and have the meanings given below:

The use of the term “about” in the present disclosure means “approximately,” and illustratively, the use of the term “about” indicates that dosages outside the cited ranges may also be effective and safe, and such dosages are also encompassed by the scope of the present claims.

“Bioavailability” refers to the extent to which an active moiety (drug or metabolite) is absorbed into the general circulation and becomes available at the site of drug action in the body.

The term “cortisol-modulating agent” means any agent possessing pharmacological activity as regulating, preventing or decreasing any pathological increase in cortisol synthesis. It also emcompasses re-balancing, or tending to re-balance, cortisol synthesis, and therefore the intensity of the cortisol effect at a certain physiological activity. The definition of “cortisol-modulating agent” as used herein can also mean that the agent possessing cortisol-modulating or -regulating or -re-balancing pharmacological activity may, if desired, be in the form of a free base, a free acid, a salt, an ester, a hydrate, an amide, an enantiomer, an isomer, a tautomer, a prodrug, a polymorph, a derivative or the like, provided the free base, free acid, salt, ester, hydrate, amide, enantiomer, isomer, tautomer, prodrug, or derivative is suitable pharmacologically, that is, effective in the present methods, combinations, kits, and compositions.

The term “cortisol-modulating-effective amount” means the amount of pharmaceutical agent effective to achieve a pharmacological effect or therapeutic improvement, leading to a clinical improvement, without undue adverse side effects, including but not limited to, improving morbidity, improving the daily life comfort, improving survival rate, more rapid recovery, or improving or eliminating symptoms and other indicators as are selected as appropriate measures by those skilled in the art, without undue adverse side effects.

As used herein, a “cortisol-mediated disease” or a “cortisol-mediated disorder” encompasses any cortisol-related disease or disorder and includes but is not limited to and a musculoskeletal, gastrointestinal, cardiovascular (including, for example, hypertension and hypercholesterolemia), endocrine and/or central nervous system (including, for example, stroke, brain, retina and/or spinal cord injuries, ischemia and reperfusion, and other brain or retinal disorders, and trauma associated with neurosurgical procedures) disease or disorder; a change in metabolism; cognitive impairment; immunosuppression; a psychiatric disorder, including depression, dementia, and mood alteration; acquired immunodeficiency syndrome; multiple sclerosis; Alzheimer's disease; Huntington's disease; epilepsy; lathyrism; amyotrophic lateral sclerosis; Parkinson's disease; and cancers, including breast cancer.

The term “derivative” refers to a compound that is produced from another compound of similar structure by the replacement of substitution of one atom, molecule or group by another. For example, a hydrogen atom of a compound may be substituted by alkyl, acyl, amino, etc., or an oxygen atom may be substituted by a nitrogen to produce a derivative of that compound.

“Drug absorption” or “absorption” refers to the process of movement from the site of administration of a drug toward the systemic circulation, for example, into the bloodstream of a subject.

An “effective amount” or “therapeutically effective amount” refers to the amount of the compound which is required to confer therapeutic effect on the treated subject.

The term “measurable serum concentration” means the serum concentration (typically measured in mmol, imol, nmol, mg, mg, or ng of therapeutic agent per ml, dl, or l of blood serum) of a therapeutic agent absorbed into the bloodstream after administration.

“Metabolism” refers to the process of chemical biotransformations of drugs in the body.

The term “pharmaceutically acceptable” is used adjectivally herein to mean that the modified noun is appropriate for use in a pharmaceutical product.

As used herein, the terms “pharmaceutically acceptable carrier” or “pharmaceutically acceptable excipients” includes any and all solvents, dispersion media, coatings, antibacterial and antifungal agents, preservative and antioxidative agents, isotonic and absorption delaying agents, and the like. The use of such media and agents for ingestible substances is well known in the art. Except insofar as any conventional media or agent is incompatible with the compositions, its use is contemplated.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasma concentration” refers to the concentration of a substance in blood plasma or blood serum.

“Plasma half-life” refers to the time required for the plasma drug concentration to decrease by 50% from its maximum concentration.

The term “prevent” or “prevention,” in relation to a cortisol-mediated disease or disorder in a subject, means no disease or disorder development if none had occurred, or no further disorder or disease development if there had already been development of the disorder or disease.

The term “prodrug” refers to a drug or compound (active principal) that elicits the pharmacological action resulting from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard, Int. J. Pharmaceutics, 37, 87 (1987) describe the evaluation of N-acylsulfonamides as potential prodrug derivatives. J. Larsen et al., Int. J. Pharmaceutics, 47, 103 (1988) describe the evaluation of N-methylsulfonamides as potential prodrug derivatives. Prodrugs are also described in, for example, Sinkula et al., J. Pharm. Sci., 64:181-210 (1975).

The term “treat” or “treatment” as used herein refers to any treatment of a disorder or disease associated with a cortisol-mediated disease or disorder, in a subject, and includes, but is not limited to, preventing the disorder or disease from occurring in a subject who may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The present invention relates to compounds that may be utilized in compositions and methods for modulating cortisol levels. The compounds of the present invention are benzoic acid derivatives represented by formula (I): 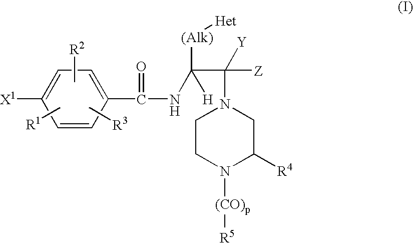
wherein:

a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;

b) X¹ is NO₂, CN, —N═O, (C₁-C₆)alkylC(O)NH—, isoxazolyl, or N(R⁶)(R⁷) wherein, R⁶ and R⁷ are individually, H, (C₁-C₆)alkyl, (C₂-C₆)alkenyl, (C₃-C₆)cycloalkyl, ((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);

c) Alk is (C₁-C₆)alkyl;

d) Y and Z are ═O, —O(CH₂)ₘO— or —(CH₂)ₘ— wherein m is 2-4, or Y is H and Z is OH or SH;

e) Het is heteroaryl or heterocycloalkyl, each optionally substituted by 1, 2 or 3 R¹, or a combination thereof, or is a bond connecting (Alk) to NH;

f) p is 0 or 1; and the pharmaceutically acceptable salts thereof.

Preferably (Alk) is (C₁-C₄)alkyl, such as —(CH₂)—, —(CH₂)₂—, —(CH₂)₃— or —(CH₂)₄—.

Preferably, 1, 2 or 3 of R¹, R² and R³ is H.

Preferably, R⁶ and R⁷ are individually H, (C₁-C₄)alkyl (C₃-C₆)cycloalkyl, (C₃-C₆)cycloalkyl(C₁-C₆)alkyl or benzyl.

Preferably, X¹ is NO₂.

Preferably, each of R⁴ and R⁵ is (C₁-C₆)alkyl or (C₃-C₆)cycloalkyl.

Preferably, 1 or 2 of R¹, R² or R³ is (C₁-C₆)alkoxy.

Preferably, p is 1.

Preferably, Z and Y together are ═O.

Preferably, Het is heteroaryl, e.g., 1H-indol-3-yl, indan-3-yl or 1H-imidazol-4-yl.

The invention also provides a pharmaceutical composition, such as a unit dosage form, comprising a compound of formula I, or a pharmaceutically acceptable salt thereof, in combination with a pharmaceutically acceptable diluent or carrier, and can optionally include stabilizers, preservatives, buffers, and absorption control agents.

Additionally, the invention provides a therapeutic method for preventing or treating a pathological condition or symptom in a mammal, such as a human, wherein the infectivity of a pathogenic agent or microorganism such as a virus or a retrovirus toward mammalian cells is implicated and inhibition of its infectivity is desired comprising administering to a mammal in need of such therapy, an effective amount of a compound of formula I, or a pharmaceutically acceptable salt thereof.

The invention provides a compound of formula I for use in medical therapy as well as the use of a compound of formula I for the manufacture of a medicament useful for the treatment of a condition amelioirated by modulation, e.g., lowering of cortisol levels, in a mammal, such as a human.

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-infectious activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl iso-butyl, sec-butyl pentyl 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; heterocycloalkyl and heterocycloalkylalkyl includes the foregoing cycloalkyl wherein the ring optionally comprises 1-2 S, non-peroxide O or N(R⁸) as well as 2-5 carbon atoms; such as morpholinyl piperidinyl, piperazinyl, indanyl, 1,3-dithian-2-yl, and the like; (C₁-C₆)alkoxy can be methoxy, ethoxy, propoxy, isopropoxy, butoxy, iso-butoxy, sec-butoxy, pentoxy, 3-pentoxy, or hexyloxy, (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl or pentafluoroethyl; hydroxy(C₁-C₆)alkyl can be alkyl substituted with 1 or 2 OH groups, such as hydroxymethyl, 1-hydroxyethyl, 2-hydroxyethyl, 1-hydroxypropyl 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy, aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), 1H-indolyl, isoquinolyl (or its N-oxide) or quinolyl (or its N-oxide).

The compounds of formula (I) wherein Y and Z are ═O (oxo), are formally N-phenacyl derivatives of heterocyclic- or heteroaryl-alpha-amino acid piperazinyl amides. Thus, methods generally applicable to peptide synthesis can be employed to prepare compounds of formula I. For example, see published PCT application WO 02/094857, U.S. Pat. Nos. 6,043,218, 6,407,211 and 5,583,108.

In general, compounds of formula (I) wherein Ar is 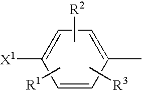
wherein X¹, R¹, R², R³, R⁴, R⁵, Het n and p are as defined above and X and Y are ═O are prepared from aminoalkyl derivatives of formula II as shown in Scheme 1, below, wherein L is Cl or Br. 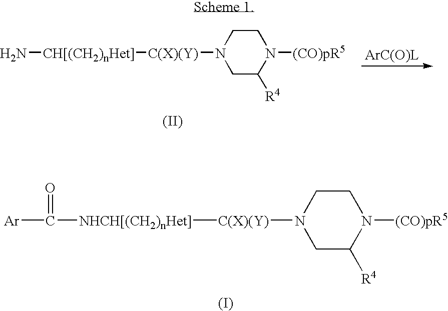
Preparation of Compounds of Formula II.

A compound of formula IIa, is prepared as shown in Scheme 2, below. 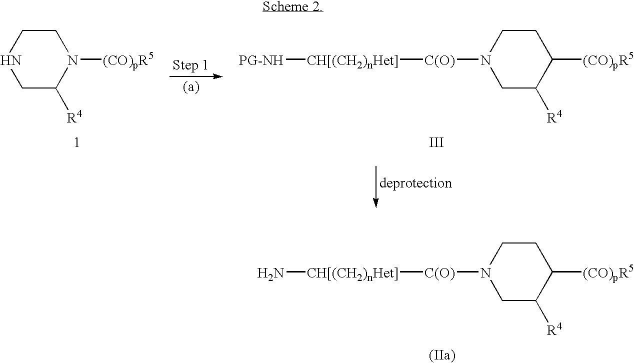
In general, compounds of formula IIa, are prepared in two steps by first converting a compound of formula I to an N-protected aminoalkyl derivative of formula III via methods (a), followed by removal of the amino protecting in III, as described below.
Preparation of Compounds of Formula III
Method (a)

In method (a), an N-protected aminoalkyl derivative of formula III where PG is an amino protecting group (e.g., tert-butoxycarboyl (BOC), benzyloxycarbonyl (CBZ), benzyl, and the like) is prepared by reacting a compound of formula 1 with a compound of formula 4:
PG-NH—CH[(CH₂)ₙHet]X (4)
where X is carboxy (—COOH) or a reactive carboxy derivative, e.g., acid halide. The reaction conditions employed depend on the nature of the X group. If X is a carboxy group, the reaction is carried out in the presence of a suitable coupling agent (e.g., N,N-dicyclohexylcarbodiimide, 1-(3-dimethylaminopropyl)-3-ethylcarbodiimide, and the like) in a suitable organic solvent (e.g., methylene chloride, tetrahydrofuran, and the like) to give an amide intermediate. If X is an acid derivative such as an acid chloride, the reaction is carried out in the presence of a suitable base such as triethylamine, pyridine in an organic solvent (e.g., methylene chloride, dichloroethane, N,N-dimethylformamide, and the like) to give an amide intermediate.

In general, compounds of formula 4 which are N-protected, heterocyclic or heteroaryl α-amino acids or are derived therefrom, are either commercially available or they can be prepared by methods well known in the field of organic chemistry.

Generally, both natural and unnatural amino acids useful in the present invention are commercially available from vendors such as Sigma-Aldrich and Bachem. Examples of natural amino acids are tryptophan and histidine. Unnatural amino acids include, 3-(indan-3-yl)-2-aminopropanoic acid, 3-(morpholin-1-yl)-2-aminopropanoic acid, 3-(piperidin-1-yl)-2-aminopropanoic acid, 3-(piperazin-1-yl)-2-aminopropanoic acid, 3-(pyridin-2-yl)-2-aminopropanoic acid, 4-(pyridin-2-yl)-2-aminobutanoic acid, 4-(imidazol-2-yl)-2-aminobutanoic acid, 4-(benzofuran-2-yl)-2-aminobutanoic acid; 3-(1,3-dithian-2-yl)-2-aminopropanoic acid and the like.

Compounds of formula 4 where X is an acid derivative, e.g., an acid chloride, can be prepared from the corresponding acids of formula 4 (X is —COOH), by chlorinating the carboxy group with a suitable chlorinating agent (e.g., oxyalyl chloride, thionyl chloride and the like) in a suitable organic solvent such as methylene chloride and the like.

Method (b)

Compounds of formula I are prepared as shown in Scheme C below by reacting a piperazine of formula 7 with a compound of formula 6, followed by the removal of the amino protecting group, utilizing the reaction conditions described in method (a) above. 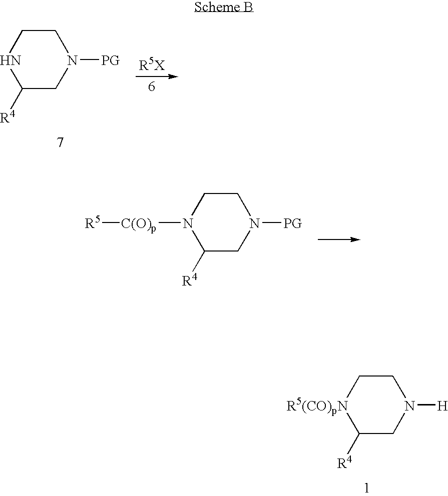
reaction conditions described in method (a) above. Method (b) is particularly suitable for preparing compounds of Formula IIa wherein R⁵X contains an amido or a carbonyl group.

In general, compounds of formula 6 which can also be used to introduce the moiety [X¹(R¹)(R²)(R³)Ph]C(O) into the compound of formula I are commercially available or can be prepared by methods well known in the art. For example, arakyl halides and arakyl acids such as benzyl bromide, 3,4-dichlorobenzyl bromide, phenylacetic acids and 2-phenylpropionic acids are commercially available. Others can be prepared from suitable starting materials such as phenylacetic acid, phenylpropanol, 2-pyridineethanol, nicotinic acid etc., by following procedures described for the synthesis of compounds of formula 4 in method (a) above.

piperazines and homopiperazines of formula 7 such as piperazine, 2 or 3-methylpiperazines and homopiperazine are commercially available. Piperazines 7 can also be prepared by following the procedures described in the European Pat. Pub. No. 0 068 544 and U.S. Pat. No. 3,267,104.

Compounds of Formula (I) where Ar is 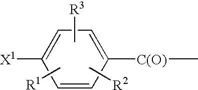
are prepared as described in Scheme C below:

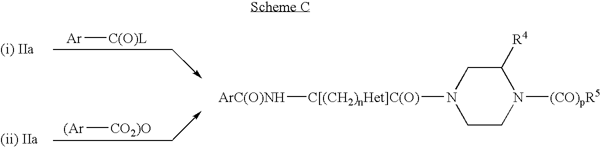

A compound of Formula (I) can be prepared, either:

(i) by reacting a compound of Formula IIa, with an acylating reagent Ar—C(O)L, wherein L is a leaving group under acylating conditions, such as a halo (particularly Cl or Br) or imidazolide. Suitable solvents for the reaction include aprotic polar solvents (e.g., dichloromethane, THF, dioxane and the like). When an acyl halide is used as the acylating agent the reaction is carried out in the presence of a non-nucleophilic organic base (e.g., triethylamine or pyridine, preferably pyridine); or

(ii) by heating a compound of formula IIa with an acid anhydride. Suitable solvents for the reaction are tetrahydrofuran, dioxane and the like; or

(iii) reacting a compound of formula IIa, or a compound of formula H₂NCH-((Alk)Het)C(O)Ot-Bu (8) with a compound of formula ArCHO in the presence of NaCNBH₄, followed by hydrolysis of the ester group, if present. Many alpha-amino acid t-butyl esters are commercially available, e.g., from Bachem.

Thus, a specific value for R¹ in formula I, above is H, (C₂-C₄)alkyl, (C₂-C₄)alkoxy or (C₃-C₆)heterocycloalkyl.

A specific value for R² is H.

A specific value for R³ is H.

A specific value for X¹ is NO₂.

A specific value for N(R⁶)(R⁷) is amino, diethyl amino, dipropylamino, cyclohexylamino, or propylamino.

A specific value for (Alk) is —(CH₂)—.

A specific value for R⁴ is CH₃.

A specific value for R⁵ is cyclopropyl.

Another preferred group of compounds are compounds of formula I which are 4N-alkanoylpiperazin-1-yl-carbonylalkylbenzamides.

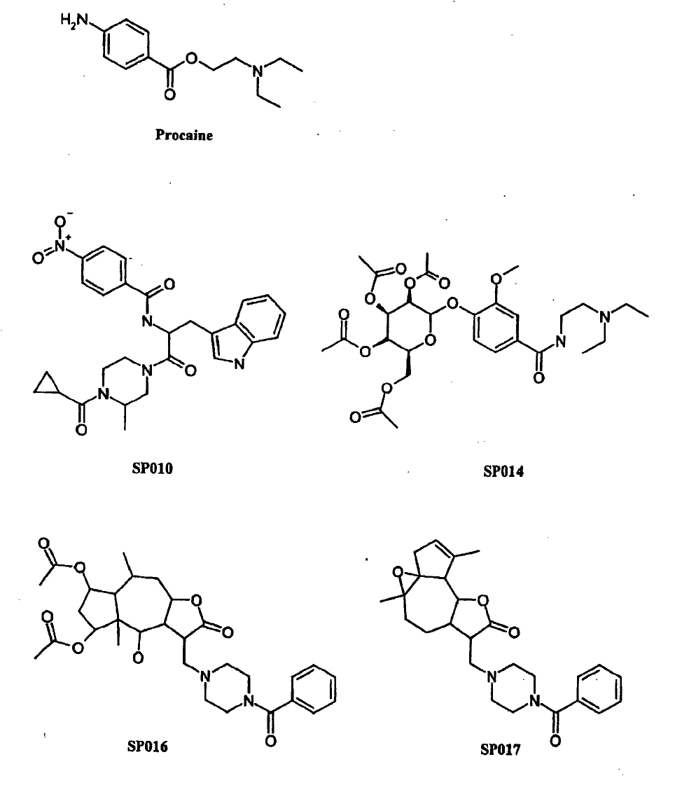

A preferred compound of the invention is SP10 (FIG. 1).

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

While not wishing to be bound by theory, it is believed that, benzoic acid derivatives represented by formula (I), as well as topical anesthetics such as procaine and procaine derivatives, for example, modulate circulating glucocorticoid levels by either inhibiting the dibutyryl cyclic AMP (dbcAMP)-induced steroid synthesis in adrenal cells by reducing HMG-CoA reductase mRNA expression and activity or by regulating the intracellular calcium concentration, such as by regulating calcium trafficking and liberation from intracellular stores. Preclinical trials performed on procaine indicate that it effectively interferes with a pathological hyperactivity of the calcium metabolism and raise of cortisol levels, the metabolic pathways described to be initiated by the intracellular infiltration of gp120.

Because procaine HCl is a nonspecific immunomodulator that can alter the normal functional expression of immunocompetent cells in vitro, it may help alleviate immunosuppresion by this more direct route. Thus, compounds of formula (I), as well as procaine and/or its bioequivalent procaine derivatives may prove effective in treating HIV-associated dementia by more than a single pathway. These compounds may prevent the cell death by directly affecting intracellular cholesterol, and therefore any steroids (including cortisol), production. Alternatively, procaine derivatives may directly target and regulate the intracellular calcium network, a network which is, as explained above, disrupted by the gp120. While these pathways differ, the mechanism of action remains the same—prevent the death of mitochondria and subsequent elevations in cortisol levels induced by the infiltration of gp120. Thus, compounds of formula (I) as well as procaine and procaine derivatives may specifically target the suspected cause of HIV-associated dementia-neuronal cell death due to elevations in cortisol levels initiated by death of mitochondria induced by increased calcium concentration and/or cortisol levels initiated by gp120 infiltration.

Benzoic acid derivatives may be chemically synthesized or derived from plant extracts and may be identified by in silico screeing of chemical and natural product databases. Several procaine derivatives that may be useful in the present invention are listed in Table 1.

TABLE 1

| | Chemical denomination | Origin |
|---|---|---|
| SP010 | 1-(4-cyclopropanecarbonyl-3-methyl- | Chemical |
| | piperazin-1-yl)-2-(1H-indol-3-yl-methyl)- | Synthesis |
| | 4-(4-nitrophenyl)-butane-1,4-dione-3-aza | |
| SP014 | Acetic acid 4,5-diacetoxy-2-acetoxymethyl- | Viburnum |
| | 6-[4-(2-diethylamino-ethylcarbamoyl)-2- | awabuki |
| | methoxy-phenoxy]-tetrahydro-pyran-3-yl | (Caprifoliaceae) |
| | ester | |
| SP016 | Acetic acid 5-acetoxy-3-(4-benzoyl- | Inula |
| | piperazin-1-yl-methyl)-4-hydroxy-4a,8- | Britanica |
| | dimethyl-2-oxo-dodecahydro-azuleno[6,5-b] | (Asteraceae) |
| | furan-4-yl ester | |
| SP017 | 3-(4-benzoyl-piperazin-1-yl-methyl)-6,6a- | Artemisia |
| | epoxy-6,9-dimethyl-3a,4,5,6,6a,7,9a,9b- | glabella |
| | octahydro-3H-azuleno[4,5-b]furan-2-one | (Asteraceae |

A compound of the present invention also includes a pharmaceutically-acceptable salt, an ester, an amide, an enantiomer, an isomer, a tautomer, a polymorph, a prodrug, or a derivative thereof. Such salts, for example, can be formed between a positively charged substituent in a compound (e.g., amino) and an anion. Suitable anions include, but are not limited to, chloride, bromide, iodide, sulfate, nitrate, phosphate, citrate, methanesulfonate, trifluoroacetate, and acetate. Likewise, a negatively charged substituent in a compound (e.g., carboxylate) can form a salt with a cation. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like. Examples of prodrugs include esters and other pharmaceutically acceptable derivatives, which, upon administration to a subject, are capable of providing compounds described above.

The compounds of the present invention are usually administered in the form of pharmaceutical compositions. These compositions can be administered by any appropriate route including, but not limited to, oral, nasogastric, rectal, transdermal, parenteral (for example, subcutaneous, intramuscular, intravenous, intramedullary and intradermal injections, or infusion techniques administration), intranasal, transmucosal, implantation, vaginal, topical, buccal, and sublingual. Such preparations may routinely contain buffering agents, preservatives, penetration enhancers, compatible carriers and other therapeutic or non-therapeutic ingredients.

The present invention also includes a pharmaceutical composition that contains the compound of the present invention associated with pharmaceutically acceptable carriers or excipients. In making the compositions of the present invention, the compositions(s) can be mixed with a pharmaceutically acceptable excipient, diluted by the excipient or enclosed within such a carrier, which can be in the form of a capsule, sachet, or other container. The carrier materials that can be employed in making the composition of the present invention are any of those commonly used excipients in pharmaceutics and should be selected on the basis of compatibility with the active drug and the release profile properties of the desired dosage form.

Illustratively, pharmaceutical excipients are chosen below as examples:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, sodium starch glycolate, crospovidone, cross-linked polyvinylpyrrolidone, croscarmellose sodium, microcrystalline cellulose, a calcium, a sodium alginate complex, clays, alginates, gums, or sodium starch glycolate, and any disintegration agents used in tablet preparations.

(c) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants such as sodium lauryl sulfate, sorbitan monooleate, polyoxyethylene sorbitan monooleate, polysorbates, polaxomers, bile salts, glyceryl monostearate, Pluronic™ line (BASF), and the like.

(e) Solubilizer such as citric acid, succinic acid, fumaric acid, malic acid, tartaric acid, maleic acid, glutaric acid sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i). Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

(j) Anti-adherents or glidants such as talc, corn starch, DL-leucine, sodium lauryl sulfate, and magnesium, calcium, or sodium stearates, and the like.

(k) Pharmaceutically compatible carrier comprises acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's The Science and Practice of Pharmacy (2000). Another discussion of drug formulations can be found in Liberman, H. A. and Lachman, L., Eds., Pharmaceutical Dosage Forms, Marcel Decker, New York, N.Y., 1980.

For treatment of a cortisol-mediated disorder, compositions of the invention can be used to provide a dose of a compound of the present invention of about 5 ng to about 1000 mg, or about 100 ng to about 600 mg, or about 1 mg to about 500 mg, or about 20 mg to about 400 mg. Typically a dosage effective amount will range from about 0.0001 mg/kg to 1500 mg/kg, more preferably 1 to 1000 mg/kg, more preferably from about 1 to 150 mg/kg of body weight, and most preferably about 50 to 100 mg/kg of body weight. A dose can be administered in one to about four doses per day, or in as many doses per day to elicit a therapeutic effect. Illustratively, a dosage unit of a composition of the present invention can typically contain, for example, about 5 ng, 50 ng 100 ng, 500 ng, 1 mg, 10 mg, 20 mg, 40 mg, 80 mg, 100 mg, 125 mg, 150 mg, 200 mg, 250 mg, 300 mg, 350 mg, 400 mg, 450 mg, 500 mg, 550 mg, 600 mg, 700 mg, 800 mg, 900 mg, or 1000 mg of a compound of the present invention. The dosage form can be selected to accommodate the desired frequency of administration used to achieve the specified dosage.

In one embodiment of the present invention, the composition is administered to a subject in an effective amount, that is, the composition is administered in an amount that achieves a therapeutically effective dose of a compound of the present invention in the blood serum of a subject for a period of time to elicit a desired therapeutic effect. Illustratively, in a fasting adult human (fasting for generally at east 10 hours) the composition is administered to achieve a therapeutically effective dose of a compound of the present invention in the blood serum of a subject from about 5 minutes after administration of the composition. In another embodiment of the present invention, a therapeutically effective dose of the compound is achieved in the blood serum of a subject at about 10 minutes from the time of administration of the composition to the subject. In another embodiment of the present invention, a therapeutically effective dose of the compound is achieved in the blood serum of a subject at about 20 minutes from the time of administration of the composition to the subject. In yet another embodiment of the present invention, a therapeutically effective dose of the compound is achieved in the blood serum of a subject at about 30 minutes from the time of administration of the composition to the subject. In still another embodiment of the present invention, a therapeutically effective dose of the compound is achieved in the blood serum of a subject at about 40 minutes from the time of administration of the composition to the subject. In one embodiment of the present invention, a therapeutically effective dose of the compound is achieved in the blood serum of a subject at about 20 minutes to about 12 hours from the time of administration of the composition to the subject. In another embodiment of the present invention, a therapeutically effective dose of the compound is achieved in the blood serum of a subject at about 20 minutes to about 6 hours from the time of administration of the composition to the subject. In yet another embodiment of the present invention, a therapeutically effective dose of the compound is achieved in the blood serum of a subject at about 20 minutes to about 2 hours from the time of administration of the composition to the subject. In still another embodiment of the present invention, a therapeutically effective dose of the compound is achieved in the blood serum of a subject at about 40 minutes to about 2 hours from the time of administration of the composition to the subject. And in yet another embodiment of the present invention, a therapeutically effective dose of the compound is achieved in the blood serum of a subject at about 40 minutes to about 1 hour from the time of administration of the composition to the subject.

Generally speaking, a patient will be administered an amount of procaine HCl that is effective to achieve a serum level commensurate with the concentrations found to be effective in vitro for a period of time effective to elicit a therapeutic effect on HIV-associated dementia. Thus, where a procaine HCl compound is found to demonstrate in vitro activity, for example, a half-maximum effective dose of 200 nM, the patient will be administered an amount of the drug that is effective to provide about a half-maximum effective dose of 200 nM concentration in vivo for a period of time that elicits a desired therapeutic effect in HIV-associated dementia. In order to measure and determine the effective amount of a compound of the present invention to be delivered to a subject, serum compound concentrations can be measured using standard assay techniques.

In one embodiment of the present invention, a composition of the present invention is administered at a dose suitable to provide a blood serum concentration with a half maximum dose of a compound of the present invention. Illustratively, a blood serum concentration of about 0.01 to about 1000 nM or about 0.1 to about 750 nM, or about 1 to about 500 nM, or about 20 to about 1000 nM, or about 100 to about 500 nM, or about 200 to about 400 nM is achieved in a subject after administration of a composition of the present invention.

Contemplated compositions of the present invention provide a therapeutic effect as the compound present invention medicates over an interval of about 5 minutes to about 24 hours after administration, enabling once-a-day or twice-a-day administration if desired. In one embodiment of the present invention, the composition is administered at a dose suitable to provide an average blood serum concentration with a half maximum dose of a compound of the present invention of at least about 1 mg/ml; or at least about 5 mg/ml, or at least about 10 mg/ml, or at least about 50 mg/ml, or at least about 100 mg/ml, or at least about 500 mg/ml, or at least about 1000 mg/ml in a subject about 10, 20, 30, or 40 minutes after administration of the composition to the subject.

The amount of the compound of the present invention necessary to elicit a therapeutic effect can be experimentally determined based on, for example, the absorption rate of the compound into the blood serum, the bioavailability of the compound, and the potency for treating the disorder. It is understood, however, that specific dose levels of the compounds present invention for any particular subject depends upon a variety of factors including the activity of the specific compound employed, the age, body weight, general health, sex, and diet of the subject (including, for example, whether the subject is in a fasting or fed state), the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of cortisol-mediated disorders or diseases in accordance with the present invention. The interrelationship of dosages for animals and humans (based on milligrams per square meter of body surface) is described by Freireich et al., Cancer Chemother. Rep. 1966, 50, 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, depending on the route of administration, the excipient usage, and the optional co-administration with other therapeutic agents.

Toxicity and therapeutic efficacy of the active ingredients can be determined by standard pharmaceutical procedures, e.g., for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Generally speaking, one will desire to administer an amount of the compound of the present invention that is effective to achieve a serum level commensurate with the concentrations found to be effective in vitro for a period of time effective to elicit a therapeutic effect. Thus, where a compound is found to demonstrate in vitro activity at, for example, a half-maximum effective dose of 200 nM, one will desire to administer an amount of the drug that is effective to provide about a half-maximum effective dose of 200 nM concentration in vivo for a period of time that elicits a desired therapeutic effect, for example, treating a cortisol-mediated disorder and other indicators as are selected as appropriate measures by those skilled in the art. Determination of these parameters is well within the skill of the art. These considerations are well known in the art and are described in standard textbooks.

Besides being useful for human treatment, the present invention is also useful for other subjects including veterinary animals, reptiles, birds, exotic animals and farm animals, including mammals, rodents, and the like. Mammal includes a primate, for example, a monkey, or a lemur, a horse, a dog, a pig, or a cat. A rodent includes a rat, a mouse, a squirrel, or a guinea pig.

For oral administration, the pharmaceutical composition can contain a desired amount of a compound of the present invention, and be in the form of, for example, a tablet, a hard or soft capsule, a lozenge, a cachet, a troche, a dispensable powder, granules, a suspension, an elixir, a liquid, or any other form reasonably adapted for oral administration. Illustratively, such a pharmaceutical composition can be made in the form of a discrete dosage unit containing a predetermined amount of the compound such as a tablet or a capsule. Such oral dosage forms can further comprise, for example, buffering agents. Tablets, pills and the like additionally can be prepared with enteric coatings.

Pharmaceutical compositions suitable for buccal or sublingual administration include, for example, lozenges comprising the compound of the present invention in a flavored base, such as sucrose, and acacia or tragacanth, and pastilles comprising the compound in an inert base such as gelatin and glycerin or sucrose and acacia.

Liquid dosage forms for oral administration can include pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

Examples of suitable liquid dosage forms include, but are not limited, aqueous solutions comprising the compound of the present invention and beta-cyclodextrin or a water soluble derivative of beta-cyclodextrin such as sulfobutyl ether beta-cyclodextrin; heptakis-2,6-di-O-methyl-beta-cyclodextrin; hydroxypropyl-beta-cyclodextrin; and dimethyl-beta-cyclodextrin;

The pharmaceutical compositions of the present invention can also be administered by injection (intravenous, intramuscular, subcutaneous). Such injectable compositions can employ, for example, saline, dextrose, or water as a suitable carrier material. The pH value of the composition can be adjusted, if necessary, with suitable acid, base, or buffer. Suitable bulking, dispersing, wetting or suspending agents, including mannitol and polyethylene glycol (such as PEG 400), can also be included in the composition. A suitable parenteral composition can also include a compound of the present invention lyophilized in injection vials. Aqueous solutions can be added to dissolve the composition prior to injection.

The pharmaceutical compositions can be administered in the form of a suppository or the like. Such rectal formulations preferably contain the compound of the present invention in a total amount of, for example, about 0.075 to about 75% w/w, or about 0.2 to about 40% w/w, or about 0.4 to about 15% w/w. Carrier materials such as cocoa butter, theobroma oil, and other oil and polyethylene glycol suppository bases can be used in such compositions. Other carrier materials such as coatings (for example, hydroxypropyl methylcellulose film coating) and disintegrants (for example, croscarmellose sodium and cross-linked povidone) can also be employed if desired.

These pharmaceutical compositions can be prepared by any suitable method of pharmaceutics, which includes the step of bringing into association the compound of the present invention and a carrier material or carriers materials. In general, the compositions are uniformly and intimately admixing the compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binding agent, lubricant, inert diluent and/or surface active/dispersing agent(s). Molded tablets can be made by molding, in a suitable machine, the powdered compound moistened with an inert liquid diluent.

Tablets of the present invention can also be coated with a conventional coating material such as Opadry™ White YS-1-18027A (or another color) and the weight fraction of the coating can be about 3% of the total weight of the coated tablet. The pharmaceutical compositions of the present invention can be formulated so as to provide quick, sustained or delayed release of the compound of the present invention after administration to the patient by employing procedures known in the art.

When the excipient serves as a diluent, it can be a solid, semi-solid or liquid material, which acts as a vehicle, carrier or medium for the compound of the present invention. Thus, the compositions can be in the form of tablets, chewable tablets, pills, powders, lozenges, sachets, cachets, elixirs, suspensions, emulsions, solutions, syrups, aerosols (as a solid or in a liquid medium), soft and hard gelatin capsules and sterile packaged powders.

In one embodiment of the present invention, the manufacturing processes may employ one or a combination of methods including: (1) dry mixing, (2) direct compression, (3) milling, (4) dry or non-aqueous granulation, (5) wet granulation, or (6) fusion. Lachman et al., The Theory and Practice of Industrial Pharmacy (1986).

In another embodiment of the present invention, solid compositions, such as tablets, are prepared by mixing a compound of the present invention with a pharmaceutical excipient to form a solid preformulation composition containing a homogeneous mixture of the compound of the present invention and the excipient. When referring to these preformulation compositions(s) as homogeneous, it is meant that the compound is dispersed evenly throughout the composition so that the composition may be readily subdivided into equally effective unit dosage forms, such as tablets, pills and capsules. This solid preformulation is then subdivided into unit dosage forms of the type described herein.

Compressed tablets are solid dosage forms prepared by compacting a formulation containing a compound of the present invention and excipients selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. A variety of materials can be used for such enteric layers or coatings, including a number of polymeric acids and mixtures of polymeric acids with such materials as shellac, cetyl alcohol and cellulose acetate.

Use of a long-term sustained release implant may be suitable for treatment of cortisol-mediated disorders in patients who need continuous administration of the compositions of the present invention. “Long-term” release, as used herein, means that the implant is constructed and arranged to deliver therapeutic levels of the compound of the present invention for at least 30 days, and preferably 60 days. Long-term sustained release implants are well known to those of ordinary skill in the art and include some of the release systems described above.

The present methods, kits, and compositions employing a compound of the present invention can also be used in combination (“combination therapy”) with another pharmaceutical agent that is indicated for treating or preventing a cortisol-mediated disorder, such as, for example, statins, ketoconazole, metyrapone, aminoglutethimide, and etomidate. When used in conjunction with the present invention, that is, in combination therapy, an additive or synergistic effect may be achieved such that many if not all of unwanted side effects can be reduced or eliminated. The reduced side effect profile of these drugs is generally attributed to, for example, the reduce dosage necessary to achieve a therapeutic effect with the administered combination.

The phrase “combination therapy” embraces the administration of a composition of the present invention in conjunction with another pharmaceutical agent that is indicated for treating or preventing a cortisol-mediated disorder in a subject (collectively “therapeutic agents”), as part of a specific treatment regimen intended to provide a beneficial effect from the co-action of these therapeutic agents for the treatment of a cortisol-mediated disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. The sequence in which the therapeutic agents are administered is not narrowly critical. “Combination therapy” also can embrace the administration of the therapeutic agents as described above in further combination with other biologically active ingredients and with non-drug therapies, such as surgery, for example. In one embodiment, a pharmaceutical composition of the present invention is combined with surgery or irradiation to treat a cortisol-mediated disorder, for example to treat Cushing syndrome with or without pituitary carcinomas.

The therapeutic agents which make up the combination therapy may be a combined dosage form or in separate dosage forms intended for substantially simultaneous administration. The therapeutic agents that make up the combination therapy may also be administered sequentially, with either therapeutic agent being administered by a regimen calling for two step administration. Thus, a regimen may call for sequential administration of the therapeutic agents with spaced-apart administration of the separate, therapeutic agents. The time period between the multiple administration steps may range from, for example, a few minutes to several hours to days, depending upon the properties of each therapeutic agent such as potency, solubility, bioavailability, plasma half-life and kinetic profile of the therapeutic agent, as well as depending upon the effect of food ingestion and the age and condition of the subject. Circadian variation of the target molecule concentration may also determine the optimal dose interval. The therapeutic agents of the combined therapy whether administered simultaneously, substantially simultaneously, or sequentially, may involve a regimen calling for administration of one therapeutic agent by oral route and another therapeutic agent by an oral route, a percutaneous route, an intravenous route, an intramuscular route, or by direct absorption through mucous membrane tissues, for example. Whether the therapeutic agent of the combined therapy are administered orally, by inhalation spray, rectally, topically, buccally, sublingually, or parenterally (for example, subcutaneous, intramuscular, intravenous and intradermal injections), separately or together, each such therapeutic agent will be contained in a suitable pharmaceutical formulation of pharmaceutically-acceptable excipients, diluents or other formulations components.

In another embodiment of the present invention, the compound for treating a cortisol-mediated disorder comes in the form of a kit or package containing one or more of the compounds of the present invention. These compounds can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the compounds of the present invention. This drug delivery system can be used to facilitate administering any of the compounds of the present invention. In one embodiment, the system contains a plurality of dosages to be to be administered daily or weekly. The kit or package can also contain other therapeutic agents utilized in combination therapy. The kits or packages also contain a set of instructions for the subject.

In one aspect, the present invention is directed to therapeutic methods of treating a cortisol-mediated condition, disease or disorder, the method comprising administration of one or more compositions of the present invention to a subject in need thereof in an amount effective at treating the condition, disease, or disorder. The dosage regimen to prevent, give relief from, or ameliorate the condition or disorder can be modified in accordance with a variety of factors. These factors include the type, age, weight, sex, diet, and medical condition of the subject and the severity of the disorder or disease. Thus, the dosage regimen actually employed can vary widely and therefore can deviate from the dosage regimens set forth herein.

The present invention also includes methods of treating, preventing, reversing, halting or slowing the progression of a cortisol-mediated disease or disorder once it becomes clinically evident, or treating the symptoms associated with, or related to the cortisol-mediated disease or disorder, by administering to the subject a composition of the present invention. The subject may already have a cortisol-mediated disease or disorder at the time of administration, or be at risk of developing a cortisol-mediated disease or disorder. The symptoms or conditions of a cortisol-mediated disease or disorder in a subject can be determined by one skilled in the art and are described in standard textbooks. The method comprises the administration a cortisol-modulating-effective amount of one or more compounds, compositions, or combinations of the present invention to a subject in need thereof.

In another aspect, the present invention is directed to therapeutic methods of treating a cortisol-mediated condition, disease or disorder, the method comprising administration of one or more compositions of the present invention to a subject in need thereof in an amount effective at decreasing hormone-induced glucocorticoid formation without affecting basal corticosteroid formation. Further still, the present invention is directed to a method of inhibiting HMG-CoA reductase mRNA expression levels without affecting basal HMG-CoA mRNA levels, comprising administering to a subject in need thereof a pharmaceutical composition comprising a cortisolmodulating-effective amount of a compound of formula (I). In yet another embodiment, the present invention is directed to a method of regulating intracellular calcium concentration levels by administering to a subject in need thereof a pharmaceutical composition comprising a cortisol-modulating-effective amount of a compound of formula (I).

It is believed that one skilled in the art, based on the description herein, can utilize the present invention to its fullest extent. All publications recited herein are hereby incorporated by reference in their entirety. The following specific examples are therefore to be construed as merely illustrative, and not limitative of the remainder of the disclosure in any way whatsoever.

### Materials and Methods

Materials

Y1 mouse adrenal tumor cells were obtained from American Type Culture Collection (Manassas, Va.) and MA-10 mouse Leydig tumor cells were given by Dr. Mario Ascoli (University of Iowa, Iowa). Mouse Hepa1-6 cells medium were obtained from American Type Culture Collection (Manassas, Va.). UT-1 cells were provided by Dr. J L Goldstein (Sothwestern University, TX). Fetal-bovine lipo-protein deficient serum (FBLPDS) was from Intracel Corporation (Frederick, Md.). F-12K (Kaign's modification of Ham's F-12) and DMEM culture media were purchased from American Type Culture Collection and DMEM/Ham's F-12 medium, horse serum, and fetal bovine serum (FBS) were purchased from InVitrogen Corporation (Carlsbad, Calif.). Antisera used: anti-20-OH-progesterone (Endocrine Sciences, Calabasas, Calif.), anti-progesterone (ICN Pharmaceuticals, Costa Mesa, Calif.), anti-P-450_{SCC} (Research Diagnostics Inc., Flanders, N.J.), anti-G3PDH (Trevigen, Inc., Gaithersburg, Md.). ³H-20a-hydroxyprogesterone, ³H-progesterone, ³H-corticosterone and ³H-mevalonolactone were purchased from PerkinElmer Life Sciences Inc. (Boston, Mass.) and ¹⁴C-HMG-CoA was obtained from Amersham Pharmacia Biotech (Buckinghamshire, England). The MTT cell proliferation assay kit was purchased from Trevigen, Inc. (Gaithersburg, Md.), the PepTag assay for nonradioactive detection of PKA kit was purchased from Promega Corporation (Madison, Wis.) and the Varian Bond-Elut NH2 columns were obtained from Chrom Tech, Inc. (Apple Valley, Minn.). Procaine chlorhydrate and compactin were obtained from chemicals were from Sigma (St. Louis, Mo.). A pharmaceutical composition comprising procaine hydrochloride, zinc sulfate heptahydrate (used to decrease the rate of absorption of procaine), ascorbic acid (used as an antioxidant), potassium benzoate (used as preservative), and disodium phosphate (“procaine-based formulation”) and a placebo of similar composition but devoid of procaine were obtained from Samaritan Pharmaceuticals, Inc. (Las Vegas, Nev.). RNA STAT-60 was from TEL-TEST, Inc. (Friendswood, Tex.). TaqMan® Reverse Transcription Reagents, random hexamers, and SYBR® Green PCR Master Mix were from Applied Biosystems (Foster City, Calif.). Cells culture supplies were purchased form GIBCO (Grand Island, N.Y.) and cell culture plasticware was from Corning (Corning, N.Y.). All other chemicals used were of analytical grade and were obtained from various commercial sources.

In Silico Screening for Procaine Derivatives

The Interbioscreen (Moscow, Russia) and Comgenex (Budapest, Hungary) databases of chemically synthesized and naturally occurring entities were screened for compounds containing structural homology with procaine using the ISIS software (Information Systems, Inc., San Leandro, Calif.). Selected compounds were screened for their ability to inhibit dbcAMP-induced steroid formation. The structure of the selected biologically active compounds, procaine and derivatives (SP010-014-016-017), are shown in FIG. 1 and the denomination, chemical name and origin for each of these compounds is shown in Table 1.

Animal Treatment

Male 80-day-old Sprague-Dawley rats were purchased form Charles River Breeding Laboratories (Wilmington, Mass.). Rats were housed at the Georgetown University Research Resources Facility under controlled light and temperature, with free access to rat chow and water. They were housed in groups of three and acclimated to their new conditions for 2 days before treatment. All experimental protocols were reviewed and approved by the Georgetown University animal care and use committee. The procaine-based formulation and placebo (both prepared by the University of Iowa School of Pharmacy, Iowa), were administered by gavage in 1 ml volume every day for a total of 8 days. Rats were sacrificed 24 hours later. Corticosterone was measured in organic extracts (ethylacetate/ether, 1:1, v/v) of the collected sera by radioimmunoassay (Amri et al., 1996) under conditions suggested by the supplier of the antisera, ICN Pharmaceuticals (Orangeburg, N.Y.).

Cell Culture

Y1 mouse adrenal tumor cells were cultured in F12K medium containing 15% horse serum, 2.5% FBS and under 5% CO₂ (Brown et al., 1992). MA-10 mouse Leydig tumor cells were cultured in DMEM/F12 medium supplemented with 5% FBS, 2.5% horse serum and under 4% CO₂ (Brown et al., 1992). Human adrenal tumor H295R cells were maintained in DMEM/F12 with 1% ITS⁺ [insulin (1 μg/ml), transferrin (1 μg/ml), selenium (I 1 μg/ml), linoeic acid (1 μg/ml), and BSA (1.25 mg/ml)], 2.5% Nuserum and 1% Penicillin-Streptomycin at 37° C., 6% CO₂ (Amri et al., 1996). Hepa1-6 mouse hepatoma cells were cultured in DMEM medium supplemented with 10% FBS and UT-1 cells were cultured in DMEM/F12 medium supplemented with 8% FBLPDS and 2% FBS plus 40_M Compactin (Chin et al., 1982).

Determination of Steroid Synthesis and Pathway Characterization

Y1 or MA-10 cells were cultured in 96-well plates (2×10⁴ cells per well) for 18 hours, and then treated with increasing concentrations of procaine HCl, a procaine-based formulation or procaine derivatives (SP compounds) for 48 hours. Culture media were then changed and cells were stimulated with 1 mM dbcAMP and the treatment for 24 to 48 hours. To assess cytochrome P-450_{SCC} activity and gene expression, culture media were then changed and cells were stimulated with 1 mM dbcAMP and incubated with procaine and/or 22R-hydroxycholesterol 10 mM for another 48 h period. To assess the role of the HMG-CoA reductase activity, culture media were then changed and cells were stimulated with 1 mM dbcAMP and incubated with procaine and/or mevalonate 10 mM for another 48 hour period. The synthesis of 20-OH progesterone and progesterone in Y1 and MA-10 cell media respectively were measured by RIA (Brown et al., 1992). H295R human adrenal tumor cells were seeded in 48-well plates at 10⁵ cells/well and incubated for 24 hours. After removal of culture media, cells were incubated in the presence of procaine or a procaine-based formulation for another 48 hour-period. At the end of the incubation time period, cells were treated with 1 mM dbcAMP for 48 hours. Cortisol levels in the media were determined by radioimmunoassay as previously described (Amri et al., 1996).

Analysis of Mitochondrial Integrity/Cell Viability

Cell viability at the end of the incubation protocol described above was assessed using the mitochondrial integrity 3-(4,5-dimethylthiazol-2-yl)-2,5-diphenyl tetrazolium bromide (MTT) assay (Trevigen, Gaithersburg, Md.). Briefly, 10 l of the MTT solution were added to the cells in 100 l medium. After an incubation period of 4 hours, 100 l of detergent were added and cells were incubated overnight at 37° C. Formazan blue formation was quantified at 600 nm and 690 nm using the Victor quantitative detection spectrophotometer (EGG-Wallac, Gaithersburg, Md.) and the results expressed as (OD₆₀₀-OD₆₉₀).

PKA Activity Measurement

Y1 cells were cultured in 6-well plates (2×10⁵ cells per well) and treated as described above for steroid biosynthesis. At the end of the incubation, cells were washed twice with PBS and proteins were extracted using an extraction buffer (25 mM tris-HCl pH 7.4, 0.5 mM EDTA, 0.5 mM EGTA, 10 mM-mercaptoethanol, 0.5 mM PMSF, 1 g/ml leupeptin, and 1 g/ml aprotinin). After centrifugation at 18,500 g for 15 minutes, the supernatants were kept for PKA activity assay. Samples were processed using the PepTag assay for non-radioactive detection of PKA activity following the manufacturer's recommendations (Promega Corporation, Madison, Wis.).

Immunoblotting

At the end of the treatment protocol described above, Y1 cells at 90% confluency were washed 2 times with PBS, sonicated 15 seconds in extraction buffer and centrifuged at 18,500 g for 15 minutes at 4° C. Pellets were resuspended in ice-cold lysis buffer (1% Nonidet 40 in extraction buffer), sonicated briefly, and incubated on ice for 1 hour. After centrifugation (22,500 g×30 min, 4° C.), the supernatant was mixed in sample buffer 6× (0.27 M SDS, 0.6 M dithiothreitol, 0.18 M bromphenol blue in 7 ml of 0.5 M Tris-HCl, pH 6.8, and 3 ml glycerol) and boiled for 5 minutes. Proteins were subjected to SDS-PAGE (4-20% gradient SDS polyacrylamide gel) and electrophoretically transferred onto nitrocellulose membranes. The transblot sheets were blocked with 5% non fat dry milk in 25 mM Tris HCl, pH 7.5, 150 mM NaCl, and 0.1% Tween 20 overnight at 4° C. Membranes were then incubated with appropriately diluted primary antibodies 1:800 for anti-P-450_{SCC} (Research Diagnostics Inc. Flanders, N.J.) and 1:200 for anti-StAR (Amri et al., 1996), and the reaction was detected by a peroxidase-conjugated secondary antibody (Santa Cruz Biotechnology, Santa Cruz, Calif.) and enhanced chemiluminescence (Amersham Life, Arlington Heights, Ill.). The densities of the appropriate bands were determined using the OptiQuant Acquisition & Analysis software (Packard BioScience).

PBR Radioligand Binding Assays

Radioligand binding assays were performed as previously described (Papadopoulos et al., 1990, J. Biol. Chem. 265, 3772-3779). In brief, cells were scrapped from culture flasks into PBS, dispersed by trituration, and centrifuged at 1200×g for 5 min. The cells were resuspended in PBS to a final concentration of 10-50 μg protein/100 μl. Saturation binding studies were performed in a final volume of 300 μl in the presence of the radioligand [³H]PK 11195 (specific activity 83.5 Ci/mmol; NEN Life Science Products) at the indicated concentrations. Nonspecific binding was determined in the presence of 6 μM of the homologous non-radioactive ligand. After 180 min incubation at 4° C., the assays were stopped by filtration through Whatman GF/C filters (Clifton, N.J.) and washed with 10 ml PBS. Bound radioactivity was determined by liquid scintillation counting. Bound [³H]PK 11195 and [³H]cholesterol were quantified by liquid scintillation spectrometry. Dissociation constants (Kd), the number of binding sites (Bmax) and Hill coefficients (nH) for PK 11195 and cholesterol were determined by Curve-Fit (Prism version 3.0, GraphPad Software Inc., San Diego, Calif.).

HMG-CoA Reductase Assay

Y1 cells in 12-well plates (1×10⁵ cells per well) were treated with increasing concentrations of procaine HCl for 48 hours. Cells were washed twice with ice-cold PBS and incubated with ice-cold assay buffer (0.1M sucrose, 40 mM KH₂PO₄, 30 mM EDTA, 50 mM KCl, 5 mM DTT, 0.25% (v/v) of Brij 96, at pH 7.4) on ice for 20 minutes. After centrifugation for 3 minutes at 14000 g (4° C.) the supernatants were collected and used for HMG-CoA reductase activity assay. The total 150 l assay mixture contained 100-200 g protein and the NADPH-generating system (2.5 mM NADP, 20 mM glucose 6-phosphate and 20 U/ml glucose 6-phosphate dehydrogenase). The reaction was started by adding substrate (¹⁴C-HMG-CoA, 0.1 Ci) and stopped after 45 minutes by adding 10-1 of HCl 6M. ³H-mevalonolactone was also added to the samples as an extraction recovery marker. After an additional 30 minutes incubation time, to allow complete lactonization of the product, the mixture was centrifuged. The supernatant was applied to Bond-Elut NH₂ column and eluted with 1 ml of toluene/acetone (3:1). The eluate was discarded and further 4 ml of toluene/acetone was applied to the column and collected in a scintillation vial for counting ¹⁴C-mevalonate and 3H-mevalonolactone signals (Berkhout et al., 1990).

In separate experiments cells were disrupted by sonication and then treated with procaine. The direct effect of the treatment on HMG-CoA reductase activity in the homogenates was determined as described above.

Real-Time Quantitative PCR (Q-PCR)

Cells cultured in 6-well plates for 18 hours were treated with increasing concentrations of procaine HCl for the indicated time period. After treatment, cells were stimulated with 1 mM dbcAMP for 24 hours. At the end of the incubation, total cell RNA was extracted using. RNA STAT-60 (Tel-Test Inc, Friendswood, Tex.) according to the manufacturer's instructions. HMG-CoA reductase mRNA was quantified by Q-PCR using the ABI Prism 7700 sequence detection system (Perlin-Elmer/Applied Biosystems, Foster City, Calif.). RT reaction was performed using TaqMan® Reverse Transcription Reagents with 1 g total RNA and random hexamers as primers for each reaction according to the manufacturer's instructions. For quantifying mouse HMG-CoA reductase mRNA with Q-PCR, the primers were designed according to GenBank Accession Number BC 019782 using PE/AB Primer Express software, which is specifically designed for the selection of primers and probes. The forward primer was 5′-CCAAGGTGGTGAGAGAGGTGTT-3′ (22 nucleotides) and reverse primer was 5′-CGTCAACCATAGCTTCCGTAGTT-3′ (23 nucleotides), respectively. The primers were synthesized by Bio-Synthesis Inc. (Lewisville, Tex.). Reactions were performed in a reaction mixture consisting of a 20 l volume solution containing 10 l SYBR® Green PCR Master Mix and 1 l primers mix (5 M each) with 2 l cDNA. The cycling conditions were: 15 sec. at 95° C. and 1 min at 60° C. for 40 cycles following an initial step of 2 min at 50° C. and 10 min at 95° C. AmpliTaq Gold polymerase was activated at 95° C. for 10 minutes. The 18S RNA was amplified at the same time and used as an internal control. To exclude the contamination of unspecific PCR products such as primer dimers, a melting curve analysis was applied to all final PCR products after the cycling protocol. Also, the PCR reactions without the RT reaction were performed for each sample in order to exclude genomic DNA contamination. PCR products were collected and run on a 3% (w/v) agarose/TAE gel to confirm the product size. The threshold cycle (Ct) values for 18S RNA and samples were calculated using the PE/AB computer software. Ct was determined at the most exponential phase of the reaction. Relative transcript levels were calculated as x=2^{Ct}, in which Ct=E−C, and E=Ctₑₓₚₑᵣᵢₘₑₙₜ−Ct_{18S}, C=Ct_{control}−Ct_{18S}.

Protein Measurement

Protein was measured using the Bio-Rad protein assay kit (Bio-Rad Laboratories, Hercules, Calif.) and bovine serum albumin as a standard.

Statistics

Statistical analysis was performed by one-way analysis of variance (ANOVA) and unpaired Student's t test using the INSTAT 3.00 package from GraphPad (San Diego, Calif.).

### EXAMPLE 1

### Synthetic Protocol for the Compound SP010

### A. [1-(1H-indol-3-ylmethyl)-2-(3-methyl-piperazin-1-yl)-2-oxo-ethyl]carbamic acid terbutyl ester (B)

Boc-L-Tryptophan (A) (4.556 g; 15 mmol) was dissolved in CH₂Cl₂ (DCM) (60 ml), 1,1′-carbonyldiimidazole (CDI) (2.513 g, 15.5 mmol) was added and then the reaction mixture was stirred at RT for 100 min. 2-Methylpiperazine (1.502 g; 15 mmol) was added and stirring was continued at RT for 6 more hours. 1,2-Dichloroethane (DCE) (15 ml) was added and the organic solution was washed with 5% aq. Na₂CO₃, 3% aq. HCl and water, respectively. The organic phase was dried over Na₂SO₄, filtered and evaporated to dryness. The residue was solidified with diethyl ether-hexane mixture to obtain the title product (B) as a white crystalline solid (3.021 g; 52%).

### B. [2-(4-cyclopropanecarbonyl-3-methyl-piperazin-1-yl)-1-(1H-indol-3-ylmethyl)-2-(3-methyl)-2-oxo-ethyl]carbamic acid terbutyl ester (C)

The piperazine derivative obtained in the previous step (B) (3.021 g; 7.82 mmol) was dissolved in DCE (30 ml). TEA (15.64 mmol; 2.81 ml) was added followed by the addition of cyclopropanecarbonyl chloride (0.77 g; 7.43 mmol; 0.674 ml). The reaction mixture was stirred at RT for 5 hours. The organic solution was extracted with 3% aq. HCl, 3% aq. Na₂CO₃ and with water, respectively. The organic phase was dried over Na₂SO₄, filtered and evaporated to dryness to obtain the desired product as a white solid (D) (3.245 g; 91%).

### C. 2-amino-1-(4-cyclopropanecarbonyl-3-methyl-piperzin-1-yl)-3-(1H-indol-3-yl)-propan-1-one (D)

The Boc-protected amino acid derivative (C) prepared in the previous step (3.254 g; 7.16 mmol) was dissolved in DCM (5 ml). TFA (8 ml) was added while cooling in an ice-water bath. The cooling bath was removed and the reaction mixture was stirred at RT for 5 hours. The mixture was evaporated to dryness, then 10% aq. NaOH (20 ml) was added to the residue while cooling in an ice-water bath. The aqueous solution was extracted with DCE (2×30 ml) and then the combined organic phase was washed with water to neutrality. The organic solution was dried over Na₂SO₄, filtered and evaporated to dryness to obtain the free amine as a light yellow solid (D) (0.787 g; 32%).

### D. N-[2-(4-cyclopropanecarbonyl-3-methyl-piperazin-1-yl)-1-(1H-indol-3-yl-methyl)-2-oxo-ethyl]-4-nitro-benzamide (SP010)

The amino-compound obtained in the previous step (D) (0.763 g; 1.62 mmol) was dissolved in DCE (30 ml), TEA (4.05 mmol; 0.565 ml) was added followed by the addition of 4-nitrobenzoyl chloride (0.256 g; 1.54 mmol). The reaction mixture was stirred at RT for 5 hours. The organic solution was extracted with 3% aq. HCL, 3% aq. Na₂CO₃ and water respectively. The organic phase was dried over Na₂SO₄, filtered and evaporated to dryness to obtain the desired product as a yellow solid (SP010) (0.79 g; 96%). The progress of every transformation reaction was checked by TLC. The identity and the purity of the final product of each step was qualified and quantified by ¹H-NMR and LC-MS spectroscopy.

### EXAMPLE 2

### Procaine and Procaine Derivatives Inhibit the dbcAMP-induced Steroid Formation in Mouse and Human Adrenal Cell Lines

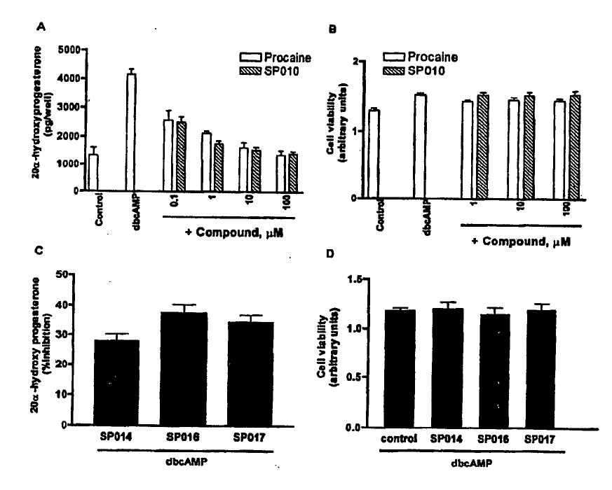

Treatment of Y1 cells with dbcAMP increased 20á-hydroxyprogesterone production by approximately 4-fold (FIG. 2A; p<0.001). Procaine and the procaine derivative SP010 decreased in a dose-dependent manner the dbcAMP-induced 201-hydroxyprogesterone production (FIG. 2A) following a dose/effect relationship. The procaine derivatives SP014, SP016, and SP017, used at 2 M concentration, reduced the dbcAMP-induced 20á-hydroxyprogesterone synthesis by Y1 cells by 30-38% (FIG. 2C). All compounds tested did not affect basal steroid formation by Y1 cells (data not shown). Moreover, none of the compounds used affected cell viability as determined using the MTT assay (FIGS. 2B & 2D).

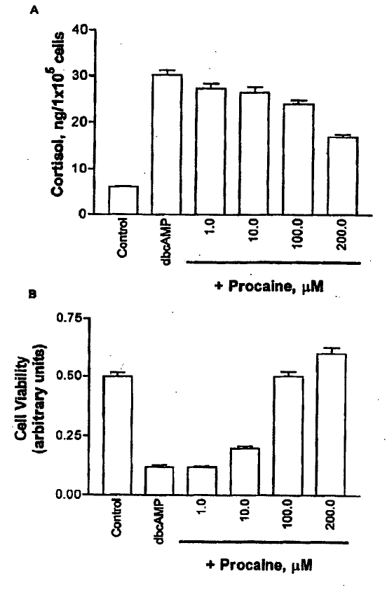

In H295R cells, dbcAMP increased cortisol synthesis by 4-fold (FIG. 3A, p<0.001). Procaine inhibited the dbcAMP-stimulated cortisol production in a dose-dependent manner (p<0.01 by ANOVA) as shown in FIG. 3A, without effecting basal cortisol production (not shown). Surprisingly, cells exposed to dbcAMP showed a dramatic decrease in cell viability, determined by the MTT assay (FIG. 3B). While not wishing to be bound by theory, cell numbers were not decreased following dbcAMP treatment suggesting that in this case, changes in MTT may reflect mitochondrial function rather than cell viability. Procaine (FIG. 3B) protected against the dbc-AMP-induced change of mitochondrial function.

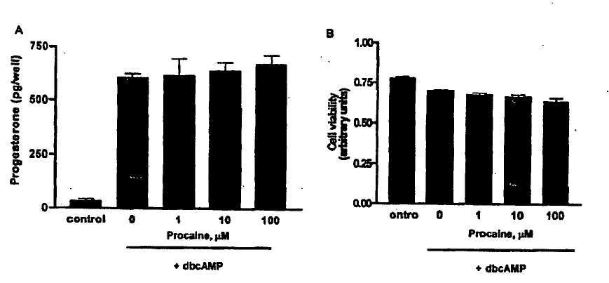

In contrast to adrenal cells, procaine did not affect the dbcAMP-induced progesterone synthesis in MA-10 mouse Leydig tumor cells (FIG. 4A). The treatment did not affect MA-10 cell viability either (FIG. 4B).

### EXAMPLE 3

### Procaine Reduces Circulating Corticosterone Levels in Male Sprague-Dawley Rats

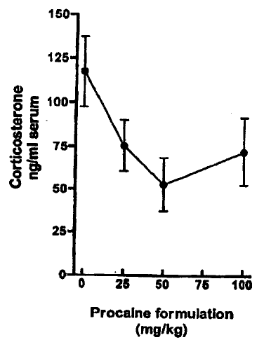

Eight days treatment of adult male rats with a procaine-based formulation reduced serum corticosterone levels by approximately 50% in a significant manner (p<0.05) as assessed by ANOVA (FIG. 5). Similar results were obtained with adult mice (data not shown).

### EXAMPLE 4

### Effect of Procaine on Various Steps of the Steroidogenic Pathway

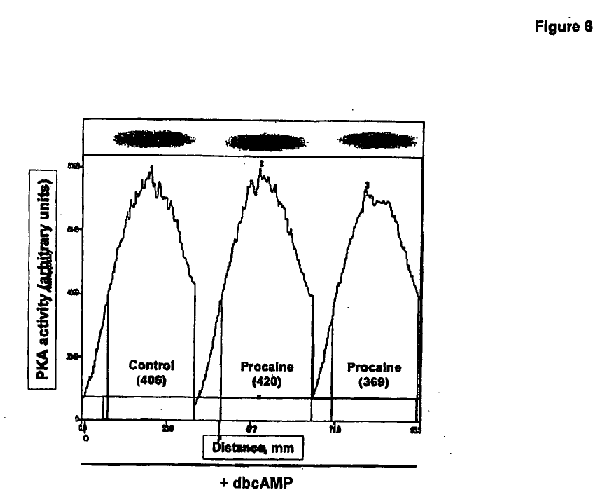

Considering the effect of procaine on the dbcAMP-stimulated steroid formation, the effect of this compound on PKA activity was investigated. PKA activity was measured using a non-radioactive detection kit based on the PKA-specific substrate, PepTag®A1 peptide (L-R-R-A-S-L-G). FIG. 6 shows that procaine at 1 M, which inhibited by 90% the dbcAMP-stimulated steroid formation (FIG. 2A), has no significant effect on the dbcAMP-stimulated PKA activity.

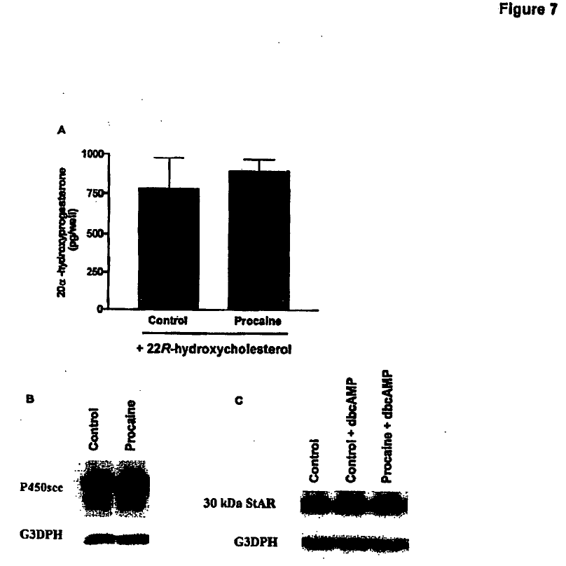

The hydrosoluble cholesterol, substrate of the P-450_{SCC}, 22R-hydroxycholesterol induced 7.5-fold increase in 20-OH progesterone formation, respectively. As shown in FIG. 2A, 1 iM procaine reduced the dbcAMP-induced steroid formation by 90%. However, procaine did not inhibit the effect of 22R-hydroxycholesterol on steroidogenesis (FIG. 7A). In addition, procaine did not modify the expression of the P-450_{SCC} enzyme as assessed by immunoblot analysis of cell extracts (FIG. 7B).

While not wishing to be bound by theory, the data presented above indicated that the effect of procaine is beyond the activation of PKA and before cholesterol metabolism to final steroid products. We examined the effect of procaine on two proteins involved in the transport of cholesterol into mitochondria, the peripheral-type benzodiazepine receptor (PBR) and the steroidogenesis acute regulatory protein (StAR) using the same 48 hour treatment protocol with procaine. These experiments showed that 1 μM procaine did not affect either the ligand binding characteristics of PBR (Bmax=27±3 pmol/mg protein and Kd=1.8 nM in control cells vs. Bmax=29±4 pmol/mg protein and Kd=1.7 nM in procaine-treated cells) nor the levels of the mature 30 kDa StAR protein (FIG. 7C) which was induced by 2.5-fold following a 3 hour dbcAMP treatment. Insight of these results, whether cholesterol synthesis itself was affected by procaine was investigated.

### EXAMPLE 5

### Procaine Inhibits the HMG-CoA Reductase Activity and mRNA Expression

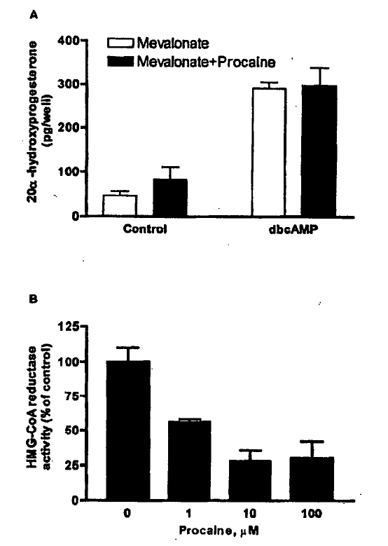

FIG. 8A shows that 1 M procaine did not inhibit the dbcAMP and mevalonate-supported 20á-hydroxyprogesterone formation, indicating that procaine may act at the level of mevalonate synthesis by the HMG-CoA reductase enzyme. HMG-CoA reductase activity was determined in Y1 cells. Procaine reduced in a dose-dependent manner HMG-CoA reductase activity in these cells (FIG. 8B). The percent inhibition for the concentration 1, 10, and 100 M were 44%, 72% and 70 respectively and the effect of the treatment was highly significant (p<0.001 by ANOVA). To assess whether the effect of procaine is due to a direct effect on the enzyme activity, Y1 cells were sonicated and treated with procaine. No direct effect of procaine on HMG-CoA reductase activity was observed (10.1±0.9 pmol/min/mg protein control versus. 9.9±0.01, 10.3±0.6, and 10.1±0.1 pmol/min/mg protein in the presence of 1, 10 and 100 μM procaine, respectively).

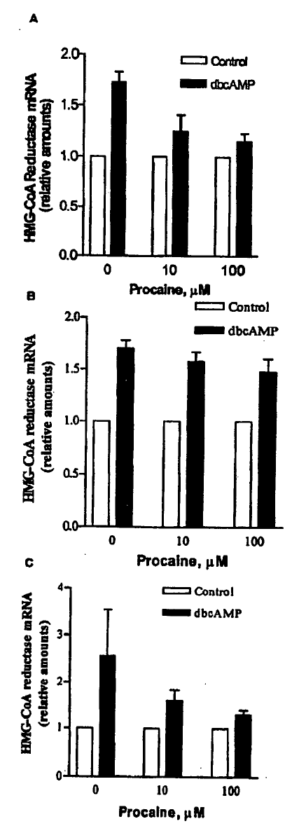

Based on these data we examined the effect of procaine on HMG-CoA reductase mRNA expression levels measured by Q-PCR and using 18SRNA as internal standard. Treatment with dbcAMP for 24 hours induced by 1.8-fold the HMG-CoA reductase mRNA expression (FIG. 9A). Pretreatment of the cells for 24 hours with procaine reduced in a dose-dependent manner HMG-CoA reductase mRNA levels (p<0.01 by ANOVA) bringing them close to the basal levels (FIG. 9A). Detailed time-course studies indicated that a 6 hour treatment with procaine was the earliest time point when the compound inhibited the dbcAMP-induced HMG-CoA reductase mRNA expression and that this effect was enhanced when cells were pre-treated for 24 hours with procaine (data not shown). Although a trend of inhibition of HMG-CoA reductase mRNA expression was seen in UT-1 cells, a Chinese hamster ovary cell clone containing high levels of HMG-CoA reductase, selected to grow in the presence of compactin, a HMG-CoA reductase inhibitor (Chin et al., 1982), this effect was not significant (FIG. 9B). However, procaine inhibited the dbcAMP-induced HMG-CoA reductase mRNA levels in Hepa1-6 mouse liver hepatoma cells (FIG. 9C) in a significant manner (p<0.01 by ANOVA).

### DISCUSSION

Procaine and procaine derivatives modulate the hormone-stimulated corticosteroid formation by adrenal cells in vitro and in vivo by, in one mechanism, reducing the levels of the rate limiting enzyme HMG-CoA reductase mRNA, leading to reduced activity, and decreased cholesterol and corticosteroid biosynthesis.

Y1 mouse adrenal tumor cells have been extensively used to understand the mechanisms underlying adrenal steroid formation. In these cells, 20-hydroxy-progesterone, an intermediate of the steroids synthesis resulting from the conversion of progesterone by 20-hydroxylase, has been used as the steroidogenic index of the cells (Mrotek and Hall, 1977; Iida et al, 1989; Brown et al., 1992). As discovered in the present invention, procaine inhibits the cAMP-induced 20-hydroxy-progesterone increase in Y1 cells without affecting basal 20-hydroxy-progesterone production by the cells. Procaine inhibits the cAMP-induced steroid synthesis at concentrations as low as 0.1 μM, and this inhibition displays a dose-response relationship over a wide-range of concentrations. This modulatory effect of procaine on the cAMP-induced steroid formation is not restricted to mouse Y1 cells but is also observed on the H295R human adrenal tumor cells, which synthesize cortisol as the main steroid product. The human adrenal tumor cells are less sensitive to procaine than the mouse adrenal cells. These results confirm and extend previous observations reporting that procaine lowered the steroidogenic effect of a cholinergic muscarinic stimulation (Hadjian et al., 1982) and of dbcAMP (Noguchi et al., 1990) on bovine adrenocortical cells. While not wishing to be bound by theory, these data together with the finding that procaine does not affect basal steroid formation by the cells evidences that procaine exerts its modulatory activity only in the presence of a stimulus.

None of the compounds tested affected adrenal cell viability, determined using the MIT assay. In contrast, in human adrenal tumor cells the treatment with dbcAMP induced a decrease in MTT levels, indicating either an effect on cell viability or an effect of the nucleotide analogue on mitochondrial diaphorase activity. This effect was not seen with Y1 cells and may be specific to H295R cells. Treatment with procaine reversed the effect of dbcAMP on mitochondrial function.

The effect of procaine was not restricted in vitro. Treatment of rats and mice for 8 days with a procaine-based formulation decreased serum corticosteroid levels by 60% compared to placebo. Thus, there is enough corticosterone remaining to support the glucocorticoid-dependent functions. 50% of the measured corticosteroid levels may reflect the normal “unstressed” condition. As the rats have not been pre-conditioned, the stress induced by being handled may be responsible for the stimulation of the corticosterone synthesis and in turn, for an increase of the plasmatic concentrations of this steroid (Kant et al., 1989). Surveys of the literature for circulating corticosterone levels in rats reveals a large variation in the reported values ranging from 4 to 40 ng/ml. Thus, in vivo treatment with procaine does not affect the basal “unstressed” adrenal function but controls the stress-induced glucocorticoid levels, thus maintaining lower “normal” circulating corticosterone levels. Procaine has been also described to decrease the release of corticotropin-releasing factor previously induced in a model of cerebral hemorrhage in rats (Plotsky et al., 1984) and to decrease the release of ACTH in a model of surgically-induced stress in the dog (Ganong et al., 1976). Such a central effect of procaine on hypothalamus and pituitary cannot be excluded to explain the decrease of the corticosterone concentrations observed in the experiments in addition to a direct effect on the adrenal cells, reinforcing the interest of procaine and its derivatives as cortisol-modulating agents.

Because procaine HCl is the ester of diethylaminoethanol and para-aminobenzoic acid and as such it can be easily hydrolyzed in the body, stable and efficient procaine derivatives exhibiting similar properties and no cell toxicity were searched. Thus, procaine derivatives were identified by in silico screening of chemical databases and tested for their ability to modulate the cAMP-corticosteroid formation. From these compounds, SP010 (Table 1) was as potent as procaine even at a concentration as low as 1 μM and displayed the same dose/response effect as procaine, suggesting a common pharmacological mechanism. However, while not wishing to be bound by theory, SP010 may also regulate cortisol levels via a regulation of the intracellular calcium concentration. The raise of the intracellular calcium concentration is a key point in the steroids synthesis-stimulating pathway and procaine has been described to modulate this calcium increase by antagonizing the activity of the ryanodine receptor (Shishan-Barmatz V. and Zchut S. (1994). Membr. Biol. 138(1): 103; Zahradinikova A. and Palade P. (1993) Biophys. J. 64(4): 991-1003). As a derivative of procaine, it is legitimate to hypothesize that SP010 exerts the same modulatory effect on the calcium pathway contributing therefore to its modulating activity on the cortisol synthesis. Procaine derivatives may also decrease the cAMP-induced expression of the ryanodine receptor RyR2 mRNA, leading to changes in intracellular calcium levels, thus contributing to its modulating activity on cortisol synthesis. A close look at the dose-response effect of procaine and SP010 on Y1 adrenocortical cells indicates that SP010 is as efficacious as procaine and procaine derivatives and maintained the same efficacy at 1, 10 and 100 iM. No effect of these compounds on basal steroid synthesis and cell viability was seen. These results suggest that the SP compounds identified based on their common procaine chemical motif are other candidates to develop drugs against pathologies due or involving increased activity of the HPA axis and thus high cortisol production.

In search of the mechanism of action of procaine on cAMP-induced adrenal steroidogenesis, the effect on the cAMP-induced PKA activity was researched. Hormone-induced PKA activity initially leads into increased cholesterol transport into mitochondria and later on in increase activity and expression of the P-450_{SCC}. The quantification of the dbcAMP-stimulated Y1 cells revealed that treatment with procaine did not affect this enzyme. In addition, procaine did not affect the rate of steroid formation by cells incubated in the presence of 22R-hydroxycholesterol, a cholesterol derivative which can cross freely the mitochondrial membranes and directly load onto the P-450_{SCC} enzyme as a substrate (Papadopoulos et al., 1990), suggesting that P450_{SCC} and other enzymes involved in the steroidogenic pathway were not affected by the procaine treatment. This result was further supported by the finding that P450_{SCC} enzyme levels were not affected by procaine. Taken together and while not wishing to be bound by theory, these data suggest that procaine and procaine derivatives affect the amount of cholesterol available for steroidogenesis. Such effect may be due either to a change in the rate of cholesterol transfer from intracellular stores into mitochondria or an effect on cholesterol synthesis. Procaine had no effect on the expression levels of PBR and StAR, the two key regulatory proteins mediating the transfer of cholesterol into mitochondria (Papadopoulos, 1998; Stocco, 2000). The finding that addition of the substrate of cholesterol synthesis mevalonate in the media together with dbcAMP resulted in abolishing the inhibitory effect of procaine on the dbcAMP-stimulated steroid formation suggested that procaine's site of action is at a step before mevalonate synthesis.

The rate-limiting enzyme in mevalonate and cholesterol biosynthesis is HMG-CoA reductase. Treatment of the cells with increasing concentrations of procaine followed by stimulation with dbcAMP resulted in the dose-dependent decrease of HMG-CoA reductase activity, assessed by the transformation of ¹⁴C-HMG-CoA into ¹⁴C-mevalonate. Maximal inhibition was achieved in the presence of 10 μM procaine. Considering the absence of a direct effect of procaine on HMG-CoA reductase activity measured in adrenal cell extracts and the fact that the effect was seen following a minimal 6 hour incubation time period, procaine may act on HMG-CoA reductase mRNA levels. Indeed, treatment of Y1 cells with dbcAMP resulted in increased HMG-CoA mRNA levels, in agreement with previous findings that cAMP and hormones regulate HMG-CoA reductase enzyme expression (Ness and Chambers, 2002; Ngo et al., 2002). Procaine inhibited in a dose-dependent manner the dbcAMP-induced HMG-CoA reductase mRNA expression levels, without affecting basal HMG-CoA mRNA levels. This finding is in agreement with the effect of procaine on the cAMP-induced steroid formation. To examine the tissue specificity of the effect of procaine on HMG-CoA mRNA expression two cell types, the UT-1 and Hepa1-6 cells, were used. UT-1 cells is a clone of Chinese hamster ovary cells (CHO-K1) that were selected to grow in the presence of compactin, a competitive inhibitor of HMG-CoA reductase. These cells have a 500-fold higher level of HMG-CoA reductase activity (Faust et al., 1982) and 100- to 1,000-fold more immunoprecipitable HMG-CoA reductase enzyme protein than normal cells (Chin et al., 1982). Hepa1-6 cells is a mouse liver hepatoma clone used because liver is the main organ in cholesterol synthesis. Treatment of both UT-1 and Hepa1-6 cells with dbcAMP induced HMG-CoA mRNA expression. Treatment of the cells with procaine resulted in the dose-dependent decrease of HMG-CoA mRNA levels. This effect was minor and not significant in the UT-1 cells but robust in the Hepa1-6 cells, suggesting that there is a tissue specificity of the effect of procaine on HMG-CoA reductase mRNA expression and activity. The finding that procaine regulates HMG-CoA reductase mRNA levels is a novel observation and the data indicating that liver cholesterol formation might be regulated by procaine is an intriguing finding that might lead to novel therapeutic applications for procaine in the field of hypercholesterolemia and related diseases. Procaine's mechanism of action via the reduction of the cAMP-induced HMG-CoA mRNA levels and SP010's mechanism of action possibly via the regulation of the calcium pathway offer alternative approaches to those currently available for regulating the HMG-CoA reductase activity. Local anesthetics, including procaine, were previously shown to affect sterol biosynthesis at a step beyond mevalonate formation (Bell and Hubert, 1980), most likely by inhibiting the cholesterol esterase (Traynor and Kunze, 1975) and cholesterol acyltransferase (Bell, 1981) enzyme activities. The data does not exclude such actions of procaine or other effects that this molecule might exert at a post-mevalonate step, effects which might be tissue specific as those described on adrenal and liver HMG-CoA reductase enzyme.

Elevated concentrations of cortisol have been reported to be associated with many diseases and to worsen the prognosis. In contrast to the detrimental effects of high levels of cortisol in the pathologies described above, maintenance of the basal cortisol levels is necessary for the maintenance of basic biological functions. Glucocorticoids regulate the metabolism of proteins, carbohydrates and lipids, and are essential to the adaptation to acute physical stressors (Munck et al, 1994). Development of compounds which block the excessive glucocorticoid synthesis without affecting the basal steroid formation has proven to be a difficult task, because it requires the identification of a modulator of an activity rather than an inhibitor. Evidence presented herein that procaine and small molecules selected for their close chemical similarity to procaine lowered the hormone-stimulated corticosteroid formation by adrenal cells in vitro and in vivo by reducing the levels of the rate limiting enzyme HMG-CoA reductase mRNA, leading to reduced activity, and decreased cholesterol and corticosteroid biosynthesis and/or by regulating intracellular calcium concentration. These compounds do not affect basal corticosteroid formation, suggesting that only pathological states of high glucocorticoid formation would be affected. Such cortisol-modulating agents may be valuable for the treatment of high cortisol diseases such as, AIDS, multiple sclerosis, AD, depression, Cushing's hypertension either alone or in combination with disease-specific therapies.

1. A method of treating a cortisol-mediated condition, disease or disorder, comprising administering to a subject in need thereof a pharmaceutical composition comprising a therapeutically effective amount of a compound of formula (I): 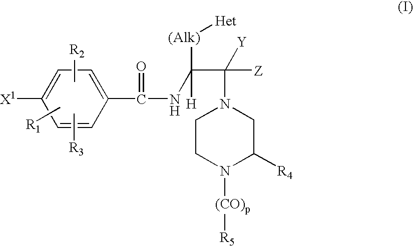
wherein:
a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;
b) X¹ is, NO₂, CN, —N═O, (C₁-C₆)alkyl(C(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);
c) Alk is (C₁-C₆)alkyl;
d) Y and Z are ═O, —O(CH₂)ₘO— or —(CH₂)ₘ— wherein m is 2-4, or Y is H and Z is OH or SH;
e) Het is heteroaryl or heterocycloalkyl, each optionally substituted by 1, 2 or 3 of R¹ or a combination thereof or is a bond connecting (Alk) to NH;
f) p is 0 or 1; and the pharmaceutically acceptable salts thereof.

2. The method of claim 1 wherein the amount is effective to treat at least one symptom of Alzheimer's disease, or vascular dementia.

3. The method of claim 1 wherein the compound of formula I is administered to a human.

4. The method of claim 1, wherein the compound of formula (I) comprises 1-(4-cyclopropanecarbonyl-3-methyl-piperazine-1-carbonyl)-(1H-indol-3-yl-methyl)-(4-nitrobenzamido)-methane.

5. A method of treating a cortisol-related condition, disease or disorder by administering to a subject in need thereof, an effective amount of acetic acid-4,5-diacetoxy-2-acetoxymethyl-6-[4-(2-diethylamino-ethylcarbamoyl)-2-methoxyphenoxy]-tetrahydro-pyran-3-yl ester.

6. A method of treating a cortisol-related condition, disease or disorder by administering to a subject in need thereof, an effective mount of acetic acid-5-acetoxy-3-(4-benzoyl-piperazin-1-yl-methyl)-4-hydroxy-4a,8-dimethyl-2-oxododecahydro-azuleno[6,5-b]furan-4-yl ester.

7. A method of treating a cortisol-related condition, disease or disorder by administering to a subject in need thereof, an effective amount of 3-(4-benzoyl-piperazin-1-yl-methyl)-6,6a-epoxy-6,9-dimethyl-3a,4,5,6,6a,7,9a,9b-octahydro-3H-azuleno[4,5-b]furan-2-one.

8. A method of treating a cortisol-related condition, disease or disorder by administering to a subject in need thereof an effective amount of procaine or a pharmaceutically acceptable salt thereof.

9. A method of inhibiting HMG-CoA reductase mRNA expression levels without affecting basal HMG-CoA mRNA levels, comprising administering to a subject in need thereof a pharmaceutical composition comprising a cortisol-modulating-effective amount of a compound of formula (I): 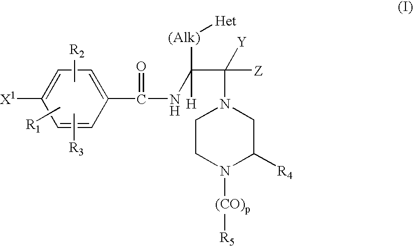
wherein:
a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkanoyloxycarbonyl; (C₁-C₆)alkylthio or (C₁-C₆)alkanoyloxy; or R¹ and R² together are methylenedioxy;
b) X¹ is, NO₂, CN, —N═O, (C₁-C₆)alkyl(C(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);
c) Alk is (C₁-C₆)alkyl;
d) Y and Z are ═O, —O(CH₂)ₘO— or —(CH₂)ₘ— wherein m is 2-4, or Y is H and Z is OH or SH;
e) Het is heteroaryl or heterocycloalkyl, each optionally substituted by 1, 2 or 3 of R¹ or a combination thereof or is a bond connecting (Alk) to NH;
f) p is 0 or 1; and the pharmaceutically acceptable salts thereof.

10. The method of claim 9, wherein the compound of formula (I) comprises (4-cyclopropanecarbonyl-3-methyl-piperazine-1-carbonyl)-(1H-indol-3-yl-methyl)-(4-nitrobenzamide)-methane.

11. A method of regulating calcium trafficking and liberation from intracellular stores leading to changes in intracellular calcium concentrations, comprising administering to a subject in need thereof a pharmaceutical composition comprising a cortisol-modulating-effective amount of a compound of formula (I): 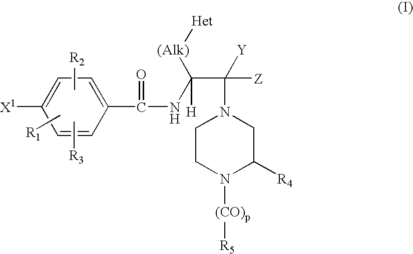
wherein:
a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;
b) X¹ is, NO₂, CN, —N═O, (C₁-C₆)alkyl(C(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, R⁶ and R⁷ together with the N to which they are attached form a 5- or 6-membered heterocyclic or heteroaryl ring, optionally substituted with R¹ and optionally comprising 1-2, S, non-peroxide O or N(R⁵);
c) Alk is (C₁-C₆)alkyl;
d) Y and Z are ═O, —O(CH₂)ₘO— or —(CH₂)ₘ— wherein m is 2-4, or Y is H and Z is OH or SH;
e) Het is heteroaryl or heterocycloalkyl, each optionally substituted by 1, 2 or 3 of R¹ or a combination thereof or is a bond connecting (Alk) to NH;
f) p is 0 or 1; and the pharmaceutically acceptable salts thereof.

12. The method of claim 11, wherein the compound of formula (I) comprises (4-cyclopropanecarbonyl-3-methyl-piperazine-1-carbonyl)-2-(1H-indol-3-yl-methyl)-4-(4-nitrophenyl)-butane-1,4-dione.

13. The method of claims 1, 9 or 11 wherein (Alk) is (C₁-C₄)alkyl, such as —(CH₂)—, —(CH₂)₂—, —(CH₂)₃— or —(CH₂)₄—.

14. The method of claims 1, 9 or 11 wherein both of R⁴ and R⁵ are (C₁-C₆)alkyl, (C₃-C₆)cycloalkyl or (C₃-C₆)cycloalkyl(C₁-C₆)alkyl, preferably (C₁-C₄)alkyl or (C₃-C₆)cycloalkyl.

15. The method of claims 1, 9 or 11 wherein 1 or 2 of R¹, R² or R³ is H or (C₁-C₆)alkoxy, preferably (C₁-C₃)alkoxy.

16. The method of claims 1, 9 or 11 wherein X and Z are ═O.

17. The method of claims 1, 9 or 11 wherein p is 1.

18. The method of claims 1, 9 or 11 where Het is 1H-indol-3-yl or imidazolin-3-yl.

19. The method of claims 1, 9 or 11 wherein the compound of formula I is administered orally to a mammal, such as a human.

20. The method of claims 1, 9 or 11 wherein the compound of formula I is administered parenterally, as by injection, infusion, inhalation or insufflation, to a mammal, such as a human.

21. The method of claims 1, 9 or 11 wherein the compound of formula (I) is administered in combination with a pharmaceutically acceptable carrier.

22. The method of claims 1, 9 or 11 wherein the carrier is a liquid, such as a solution, suspension or gel.

23. The method of claims 1, 9 or 11 wherein the carrier is a solid.

24. The method of claims 1, 9 or 11 wherein the compound of formula I is N-[2-((4-cyclopropylcarbonyl)-3-methylpiperazin-1-yl)-1-(1H-indol-3-yl-methyl)-2-(oxo)ethyl]-4-nitrobenzamide.
